(19) United States
(12) Patent Application Publication
(10) Pub. No.: US 2024/0174596 A1
(43) Pub. Date: May 30, 2024

(54) (S)-3-AMINO-4,4-DIHALOCYCLOPENT-1-ENECARBOXYLIC ACID AS SELECTIVE INACTIVATORS OF HUMAN ORNITHINE AMINOTRANSFERASE

(71) Applicant: Northwestern University, Evanston, IL (US)

(72) Inventors: Richard B. Silverman, Evanston, IL (US); Sida Shen, Evanston, IL (US)

(21) Appl. No.: 18/548,784

(22) PCT Filed: Mar. 3, 2022
(86) PCT No.: PCT/US22/18753

§ 371 Date: Sep. 1, 2023

Related U.S. Application Data
(60) Provisional application No. 63/156,147, filed on Mar. 3, 2021.

(51) Int. Cl. C07C 229/48; A61K 31/196

(52) U.S. Cl. CPC C07C 229/48; A61K 31/196; C07C 2601/10

(57) ABSTRACT

Disclosed are amino, fluoro-substituted cyclopentene carboxylic acid compounds. The disclosed compounds and compositions thereof may be utilized in methods for modulating human ornithine δ-aminotransferase (hOAT) activity, including methods for treating diseases or disorders associated with to hOAT activity or expression such as cell proliferative diseases and disorders.

### CROSS-REFERENCE TO RELATED APPLICATIONS

This application claims benefit of priority to U.S. Patent Application Ser. No. 63/156,147, filed Mar. 3, 2021, the contents of which is incorporated by reference in its entirety.

### STATEMENT REGARDING FEDERALLY SPONSORED RESEARCH OR DEVELOPMENT

This invention was made with government support under DA030604 awarded by the Department of Health and Human Services, National Institutes of Health, and National Institute on Drug Abuse. The government has certain rights in the invention.

### BACKGROUND

Human ornithine δ-aminotransferase (hOAT; EC2.6.1.13) is a pyridoxal-5′-phosphate (PLP)-dependent enzyme that catalyzes two coupled transamination reactions, converting L-ornithine (L-Orn) to afford L-glutamate-γ-semialdehyde (L-GSA) in the first half-reaction with the generation of L-glutamate (L-Glu) from α-ketoglutarate (α-KG) in the second half-reaction.¹ The afforded intermediate L-GSA is a spontaneous equilibrium species of Δ1-pyrroline-5-carboxylate (P5S), while P5C can, in turn, afford L-proline catalyzed by P5C reductases (PYCRs).² On the other hand, the generated L-Glu can also be converted to P5S by pyrroline-5-carboxylate synthase (P5CS), thus participating in proline metabolism.² Proline biosynthesis was identified as the most substantially altered amino acid metabolism in human tumor tissues of hepatocellular carcinoma (HCC), featured by accelerated proline consumption, hydroxyproline accumulation, and correlation with increased α-fetoprotein (AFP) levels and poor prognosis in HCC.³ In addition, glutamine synthetase (GS) catalyzes L-Glu's conversion to L-glutamine (L-Gln).⁴ L-Gln is highly required by cancer cells to support anabolic processes, thus promoting cellular proliferation.

HCC is the predominant liver malignancy and ranks among the most common leading causes of cancer-associated mortality worldwide. Previous work identified the OAT gene as one of the seven overexpressed genes in the spontaneous HCC-developing livers from Psammomys obesus (sand rat) identified by DNA microarray analysis.⁵ Moreover, the treatment (0.1 and 1.0 mg/kg; PO) of selective hOAT mechanism-based inactivator (MBI) BCF3 (1) remarkably decreased the serum AFP levels and inhibited tumor growth in an HCC mouse model,⁵ underscoring the antitumor effects of pharmacological selective HOAT inhibition. MBI is a type of molecule that initially acts as an alternative substrate of the target enzyme and is then transformed into active species that can further inactivate the enzyme through specific covalent modification, tight-binding electrostatic interaction, or any other functionally irreversible inhibition.⁶⁻⁷ MBIs are typically unreactive before the initial bonding with the active site of the target enzyme, thereby usually exhibiting significant target specificity and selectivity.⁸ Overall, hOAT can be considered as a potential therapeutic target for HCC, and selectively inactivating hOAT may provide a novel opportunity to discover effective HCC treatment.

However, the major challenge for discovering selective MBI of hOAT is to overcome the irreversible inhibition on other aminotransferases,⁶ especially γ-aminobutyric acid aminotransferase (GABA-AT) that has a high structural similarity with hOAT.¹ There are only two significant differences in the active site pocket of their homodimer structures: Tyr85 and Tyr55 in hOAT are replaced by Ile72 and Phe351* in GABA-AT, respectively.¹ Moreover, Ile 72 and Phe351* are responsible for the slightly narrower but more hydrophobic active site of GABA-AT relative to hOAT. In contrast, the hydroxyl group of Tyr55 serves as a hydrogen bond acceptor to interact with the charged C-2 amino group of substrates, while Tyr85 is a significant determinant of substrate specificity and conformationally flexible to adopt bulky substrates.¹

According to the high similarity between these two aminotransferases, a preliminary screening against hOAT was carried out previously using stock GABA-AT inhibitors.⁵ A cyclopentane-based analog, termed BCF3 (1), bearing bis(trifluoromethyl) group as its warhead, was identified to be a selective MBI of hOAT while only showing millimolar reversible inhibition on GABA-AT. The recent mechanistic studies have revealed that one of its trifluoromethyl groups occurs fluoride ion elimination, leading the ligand to covalently modify the catalytic Lys292 through the conjugate addition.⁹⁻¹⁰ It is considered that the steric bulky bis(trifluoromethyl) group may not be easy to access the relatively narrower pocket of GABA-AT, influencing the initial binding pose between the ligand and the enzyme, which may be responsible for its reversible inhibition on GABA-AT. BCF3 has been demonstrated to be effective in vivo as abovementioned⁵ and is being investigated in extensive IND-labelling toxicity assessment and efficacy studies in HCC patient-derived xenografted (PDX) models. Based on a similar strategy, an enlarged the ring system was tested and it was further discovered that a cyclohexene-based analog WZ-2-051 (2) bearing a difluoro group.¹¹ WZ-2-051 exhibited a 23-fold improvement in inactivation efficiency (defined by the k_{inact}/K₁ ratio) against hOAT compared to BCF3 while showing 13.3-fold selectivity over GABA-AT. The subsequent mechanistic studies have disclosed that WZ-2-051 undergoes two-step fluoride ion elimination and finally inactivates hOAT through an addition-aromatization mechanism.¹¹ An additional example of selective hOAT inactivator is 5-FMOrn (3) inspired by the structure of hOAT substrate L-Orn and the structure of non-selective GABA-AT inactivator AFPA,¹² inactivates hOAT via an enamine pathway by forming a ternary adduct.¹³

It should be noted that the α-amino group of 5-FMOrn forms a strong hydrogen bond with the phenol group of Tyr55 in the hOAT crystal complex (PDB entry 2OAT).¹⁴ Moreover, it was also observed that hydrogen bonds between their carboxylate groups and Tyr55 in the hOAT crystal complex with BCF3 (PDB entry 6OIA) and WZ-2-051 (PDB entry 6V8C),^{9,11} thereby demonstrating that the interaction with Tyr55 plays a significant in ligand specificity and selectivity. However, among the published hOAT inactivators, only BCF3 demonstrates potent irreversible inhibition on hOAT, weak reversible inhibition on GABA-AT (Kᵢ=4.2 mM), and no inhibition on aspartate aminotransferase (Asp-AT) and alanine aminotransferase (Ala-AT) up to 4 mM,⁵ leading to promising hOAT selectivity. Therefore, discovering novel selective MBIs of hOAT will facilitate the investigation of hOAT inactivators as potential therapeutic approaches for HCC.

In 2000, (1R,4S)-4-amino-3,3-difluorocyclopentanecarboxylic acid (4) was found to be a reversible inhibitor against GABA-AT (Kᵢ=0.19 mM).¹⁵ Fifteen years later, it was further demonstrated to be an hOAT inactivator.⁵ However, compound 4 exhibits high binding affinity (K₁=7.8 mM) towards hOAT and a low maximum rate of inactivation (k_{inact}=0.02 min⁻¹) against hOAT, eventually causing a modest inactivation efficiency (k_{inact}/K₁=0.003 min⁻¹mM⁻¹), which limits us to elucidate its inactivation and turnover mechanisms.

The present technology provides for novel cyclopentene-based analogs, including SS-1-148 (6) by incorporating an additional double bond into its cyclopentane ring system of 4, which are demonstrated to be a highly potent and selective hOAT inactivator. Mechanistic studies utilizing crystallization, protein and molecular mass spectrometry, transient state measurements, and computational simulations to reveal a novel non-covalent inactivation mechanism of SS-1-148.

### SUMMARY

Disclosed are compounds, compositions and related methods of use for the selective inhibition of human ornithine δ-aminotransferase (hOAT). The disclosed compounds, compositions, and methods can be utilized to treat diseases and disorders associated with human omnithine δ-aminotransferase (hOAT).

The disclosed compounds may be described as substituted cyclopentene compounds. In particular, the disclosed compounds may be described as amino, halo-substituted cyclopentene carboxylic acid compounds. The disclosed compounds and compositions thereof may be utilized in methods for modulating human ornithine δ-aminotransferase (hOAT) activity, including methods for treating diseases or disorders associated with hOAT activity or expression such as cell proliferative diseases and disorders.

The disclosed compounds may be directed to a compound of the following formula or a dissociated form, a non-protonated form, a zwitterion form, or a salt thereof:

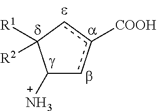

wherein a double bond is present between the a and & carbons or between the α and β carbons, and

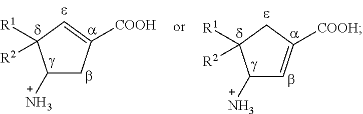

wherein each of R¹ and R² is independently selected from a halogen such as F, Cl, Br, and I.

As indicated, the disclosed compounds may be protonated, for example to form an ammonium moiety, optionally where the compound is present as a salt. The disclosed compounds also may be non-protonated and/or dissociated, for example, where the carboxylic acid moiety is dissociated to from a carboxylate moiety, optionally where the compound is present as a salt. The disclosed compounds may be in zwitterionie form where the compound comprises a protonated ammonium moiety and a dissociated carboxylate moiety, optionally where the compound is present as a salt.

The disclosed compounds and compositions may be utilized in methods for modulating human ornithine δ-aminotransferase (hOAT) activity. Such methods can comprise providing a compound as disclosed herein, such as a compound of the following formula or a dissociated form, a zwitterion form, or a salt thereof, and contacting hOAT with the compound:

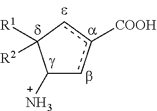

wherein a double bond is present between the α and ε carbons or between the α and β carbons, and wherein each of R¹ and R² is independently selected from a halogen such as F, Cl, Br, and I.

In certain embodiments, the disclosed methods may be directed to reducing activity of an hOAT expressed by a cancer, which may include but is not limited to hepatocellular cancer (HCC) and non-small cell lung cancer (NSCLC), or other cancers that express or overexpress hOAT. Such a method can comprise providing a compound as disclosed herein, such as a compound of the following formula or a dissociated form, a non-protonated form, a zwitterion form, or a salt thereof, and contacting the cancer with the compound:

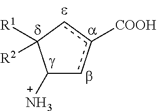

wherein a double bond is present between the α and ε carbons or between the α and β carbons, and wherein each of R¹ and R² is independently selected from a halogen such as F, Cl, Br, and I.

In certain embodiments, the disclosed methods may be directed to treating a cell proliferative disease or disorder in a subject in need thereof Suitable cell proliferative diseases and disorders may include cancers that express or overexpress hOAT such as, but not limited to, hepatocellular carcinoma (HCC) and non-small cell lung cancer (NSCLC). Such a method can comprise administering to such a subject in need thereof a compound of the following formula or a dissociated form, a non-protonated form, a zwitterion form, or a salt thereof.

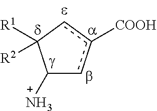

wherein a double bond is present between the α and ε carbons or between the α and β carbons, and wherein each of R¹ and R² is independently selected from a halogen such as F, Cl, Br, and I.

The compounds disclosed herein are without stereochemical or configurational limitation and encompass all stereochemical or configurational isomers, unless stereochemical or configurational limitations are indicated. As illustrated and discussed below, such compounds and/or their intermediates are available as single enantiomers, racemic mixtures from which isomers can be resolved, or diastereomers from which the corresponding enantiomers can be separated Accordingly, any stereocenter can be (S) or (R) with respect to any other stereocenter(s). As another separate consideration, various compounds can be present as an acid or base salt, either partially or fully protonated, for example at the amino group to form an ammonium moiety, and/or either partially or fully dissociated, for example at the carboxyl group to form a carboxylate substituent or moiety. In certain such embodiments, with respect to an ammonium substituent or moiety, the counter ion can be a conjugate base of a protic acid. In certain such or other embodiments, with respect to a carboxylate substituent or moiety, the counter ion can be an alkaline, alkaline-earth or ammonium cation. Further, it will be understood by those skilled in the art that any one or more the compounds disclosed herein can be provided as part of a pharmaceutical composition comprising a pharmaceutically-acceptable carrier component for use in conjunction with a treatment method or medicament.

In certain embodiments, the disclosed methods are directed to a disease or disorder associated with hOAT activity and/or expression or overexpression, including cell proliferative diseases and disorders such as cancers associated with hOAT activity and/or expression or overexpression. Suitable diseases and disorders may include, but are not limited to cell proliferative diseases and disorders, which may include but are not limited to hepatocellular carcinoma (HCC) and non-small cell lung cancer (NSCLC) in a human subject in need of such a treatment. In certain embodiments, such a compound can be provided as part of a pharmaceutical composition.

In certain embodiments, the disclosed methods are directed to reducing or modulating activity of a human ornithine δ-aminotransferase expressed by a cancer (e.g, hepatocellular carcinoma (HCC) and non-small cell lung cancer (NSCLC)). Such a method can comprise providing a compound of the sort discussed above or described elsewhere herein, and contacting such a compound with a cellular medium comprising a cancer expressing a human ornithine δ-aminotransferase with an amount of such a compound effective to reduce human ornithine δ-aminotransferase activity. In certain embodiments, such a compound can be provided as part of a pharmaceutical composition. Regardless, such contact can be in vitro or in vivo.

More generally, the disclosed methods may be directed to inhibiting or inactivating a human ornithine δ-aminotransferase. Such a method can comprise providing a compound of the sort discussed above or described below, whether or not part of a pharmaceutical composition, and administering an effective amount of such a compound for contact with a human ornithine δ-aminotransferase. Such contact can be, as would be understood in the art, for experimental and/or research purposes or as may be designed to simulate one or more in vivo or physiological conditions. Such compounds can include but are not limited to those illustrated by the following examples, referenced figures, incorporated references and/or accompanying synthetic schemes. In certain such embodiments, such a compound and/or combination thereof can be present in an amount at least partially sufficient to inhibit hOAT, cell proliferation and/or tumor growth.

### BRIEF DESCRIPTION OF THE FIGURES

FIG. 1. Coupled transamination reactions of hOAT and related metabolic pathways (A); Structures of hOAT mechanism-based inactivators BCF3 (1), WZ-2-051 (2), and 5-FMOrn (3)(B).

FIG. 2. The active site comparison of hOAT (A; PDB entry 1OAT) and GABA-AT (B; PDB entry 1OHV). C) The structures of hOAT inactivators 4-6.

FIG. 3. Primary metabolites of SS-1-148 in hOAT (A) and GABA-AT (B).

FIG. 4. Crystal structures of hOAT resulting from soaking experiment (A, PDB entry 7LK1) and cocrystallization (B, PDB entry 7LK0) with compound SS-1-148. SS-1-148 soaking structure is shown in two conformations: one in which the carboxylate group interacts with Tyr55 (conformation A) and the other in which carboxylate forms a salt bridge with Arg413 (conformation B). For this specific chain, the occupancies of conformers are 0.51 (conformation A) and 0.49 (conformation B). hOAT residues and SS-1-148 are in stick representation. H-Bonding distances between atoms in Ångstroms (Å) are shown as dashed lines.

FIG. 5. Denaturing, intact (A) and native (B) protein mass spectrometry of hOAT inactivated by SS-1-148.

FIG. 6. Transient state absorption changes observed at 275, 420, and 560 nm for hOAT reacting with SS-1-148. OAT (12.7 μM final) was mixed with SS-1-148 (126, 251, 502, 1004, 2008, 4016 μM), and CCD spectra were collected for the timeframe 0.009-49.2 seconds. (A) The data observed at 420 nm fit to a linear combination of three exponential terms according to equation (3) described in the supporting information. The arrow indicates the trend observed in amplitude for increasing inhibitor concentration. (B) The observed rate constant dependence of the first phase observed at 420 nm fit to equation (5) described in the supporting information. The values for k₂ and k₃ indicated are the average values obtained from the fit in FIG. 5A. The fit is shown in red dashes. (C) The data observed at 560 nm. The curved arrow indicates the trend observed in amplitude for increasing inhibitor concentration. These data were fit to a linear combination of two (1004, 2008, and 4016 μM) or three (126, 251, and 502 μM) exponential terms according to equation (4) described in the supporting information. The fit is shown in red dashes. (D) The data observed at 275 nm over 2000 sec obtained in the presence of 8032 μM SS-1-148 fit to a linear combination of two exponential terms according to equation (4) in the supporting information. The fit is shown in red dashes.

FIG. 7. Partial deconvolution by singular value decomposition (SVD) of transient state absorption changes observed for hOAT reacting with SS-1-148. hOAT (6.94 μM; final concentration) was reacted in a stopped-flow spectrophotometer with SS-1-148 (1040 μM; final concentration) at 10° C. To obtain time resolution sufficient to analyze kinetic rates spanning four orders of magnitude, a composite CCD absorbance dataset was prepared spanning 250-800 nm and 0.0137-9843 sec by splicing together averaged short and long time-frame datasets. These data were fit to a linear irreversible four-step model in which the rate constants were constrained to those determined from single-wavelength analyses (FIG. 6) Deconvoluted composite spectra derived from SVD analysis (A). The species concentration profile based on the rate constants used to fit the dataset (B). A three-dimensional depiction of a subset of spectra from the dataset analyzed (C).

FIG. 8. Theoretical pKₐ calculations of the hydrogen at the C_{γ} position using the DFT/B3LYP method (A) and electron density maps coded to the electrostatic potential of intermediates and ESP charges of C_{δ} and C_{4′} positions (B).

FIG. 9. Titration of hOAT with SS-1-148. The loss of enzyme activity was measured as a function of the ratio of inactivation to enzyme concentration. Linear regression was used on the linear portion of the curves to obtain the X-intercept, which was the turnover number (partition ratio=turnover number−1).

FIG. 10. Time-dependent dialysis of hOAT partially or fully inhibited by varying concentrations of SS-1-148.

FIG. 11 Polder map of SS-1-148 soaking intermediate at 4σ level.

FIG. 12. Polder map of SS-1-148 cocrystal at 4σ level.

FIG. 13. Native, unmodified hOAT examined by native MS. Left: untreated hOAT: Right: untreated hOAT with collisional dissociation energy applied (NCE: 15).

FIG. 14. Gibb's free energy for M7 and related tautomeric forms.

FIG. 15. Primary metabolites of 4-6 in hOAT. (A) Primary metabolite of 4 in hOAT. (B) Primary metabolite of 5 in hOAT. (C) Primary metabolite of SS-1-148 (6) in hOAT.

### DETAILED DESCRIPTION

The disclosed subject matter may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the claimed subject matter and does not pose a limitation on the scope of the claimed subject matter.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to.” “at least.” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, a “subject in need thereof” may include a human and/or non-human animal. A “subject in need thereof” may include a subject having a disease or disorder associated with human ornithine δ-aminotransferase (hOAT) activity. A “subject in need thereof” may include a subject having a cell proliferative disease or disorder, which may include, but is not limited to hepatocellular carcinoma (HCC)

### Chemical Entities

New chemical entities and uses for chemical entities are disclosed herein. The chemical entities may be described using terminology known in the art and further discussed below.

As used herein, a dash “−” an asterisk “*” or a plus sign “+” may be used to designate the point of attachment for any radical group or substituent group.

The term “alkyl” as contemplated herein includes a straight-chain or branched alkyl radical in all of its isomeric forms, such as a straight or branched group of 1-12, 1-10, or 1-6 carbon atoms, referred to herein as C1-C12 alkyl, C1-C10-alkyl, and C1-C6-alkyl, respectively.

The term “alkylene” refers to a diradical of straight-chain or branched alkyl group (i.e., a diradical of straight-chain or branched C₁-C6 alkyl group). Exemplary alkylene groups include, but are not limited to —CH₂—, —CH₂CH₂—, —CH₂CH₂CH₂—, —CH(CH₃)CH₂—, —CH₂CH(CH₃)CH₂—, —CH(CH₂CH₃)CH₂—, and the like.

The term “halo” refers to a halogen substitution (e.g., —F, —Cl, —Br, or —I). The term “haloalkyl” refers to an alkyl group that is substituted with at least one halogen. For example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxy” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C2-C12-alkenyl, C2-C10-alkenyl, and C2-C6-alkenyl, respectively.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C2-C12-alkynyl, C2-C10-alkynyl, and C2-C6-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C4-8-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido or carboxyamido, amidino, amino, aryl, arylalkyl, azido, carbamate, carbonate, carboxy, cyano, cycloalkyl, ester, ether, formyl, halo, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloheteroalkyl” refers to a monovalent saturated cyclic, bicyclic, or bridged cyclic hydrocarbon group of 3-12, 3-8, 4-8, or 4-6 carbons in which at least one carbon of the cycloalkane is replaced with a heteroatom such as, for example, N, O, and/or S.

The term “cycloalkylene” refers to a cycloalkyl group that is unsaturated at one or more ring bonds.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number oring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido or carboxyamido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i e., it is unsubstituted.

The term “aryl” is art-recognized and refers to a carbocyclic and/or heterocyclic aromatic group. Representative aryl groups include phenyl, naphthyl, anthracenyl, pyridinyl, quinolinyl, furanyl, thionyl, and the like. The term “aryl” includes polycyclic ring systems having two or more carbocyclic rings in which two or more carbons are common to two adjoining rings (the rings are “fused rings”) wherein at least one of the rings is aromatic and, e.g, the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido or carboxyamido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C3-C7 heterocyclyl group refers to a saturated or partially unsaturated 3- to 7-membered ring structure containing one to four heteroatoms, such as nitrogen, oxygen, and sulfur The designation “C3-C7” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines (e.g., mono-substituted amines or di-substituted amines), wherein substituents may include, for example, alkyl, cycloalkyl, heterocyclyl, alkenyl, and aryl.

The terms “alkoxy” or “alkoxyl” are art-recognized and refer to an alkyl group, as defined above, having an oxygen radical attached thereto. Representative alkoxy groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “oxo” refers to a divalent oxygen atom —O—.

The term “carboxy” or “carboxyl” as used herein refers to the radical —COOH or its corresponding salts, e.g. —COONa, etc. A carboxy alkyl ester refers to a compound having a moiety —C(O)O—R, where R is alkyl.

The term “carboxamido” as used herein refers to the radical —C(O)NRR′, where R and R′ may be the same or different. R and R′, for example, may be independently hydrogen, alkyl, aryl, arylalkyl, cycloalkyl, formyl, haloalkyl, heteroaryl, or heterocyclyl.

The term “amide” or “amido” or “amidyl” as used herein refers to a radical of the form —R¹C(O)N(R²)—, —R¹C(O)N(R²)R³—, —C(O)NR²R³, or —C(O)NH₂, wherein R¹, R² and R³, for example, are each independently hydrogen, alkyl, alkoxy, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may be isomeric. In some embodiments, the disclosed compounds may be isomerically pure, wherein the compounds represent greater than about 99% of all compounds within an isomeric mixture of compounds. Also contemplated herein are compositions comprising, consisting essentially of, or consisting of an isomerically pure compound and/or compositions that are isomerically enriched, which compositions may comprise, consist essential of, or consist of at least about 50%, 60%, 70%, 80%, 90%. 95%, 96%, 97%, 98%, 99%, or 100% of a single isomer of a given compound.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” or “+” or “−” depending on the configuration of substituents around the chiral or stereogenic carbon atom and or the optical rotation observed. The disclosed compounds encompasses various stereo isomers and mixtures thereof. Stereoisomers include enantiomers and diastereomers. Mixtures of enantiomers or diastereomers may be designated (±)” in nomenclature, but the skilled artisan will recognize that a structure may denote a chiral center implicitly It is understood that graphical depictions of chemical structures, e.g., generic chemical structures, encompass all stereoisomeric forms of the specified compounds, unless indicated otherwise. Also contemplated herein are compositions comprising, consisting essentially of, or consisting of an enantiopure compound and/or compositions that are enantiomer enriched, which compositions may comprise, consist essential of, or consist of at least about 50%, 60%, 70%, 80%, 90%, 95%, 96%, 97%, 98%, 99%, or 100% of a single enantiomer of a given compound (e.g., at least about 95% of an R enantiomer of a given compound).

Various non-limiting embodiments of the disclosed compounds and methods of use can be considered with an understanding of a catalytic mechanism of OAT and mechanism of inactivation of GABA-AT and OAT. In a some embodiments, the disclosed subject matter relates to one or more OAT inhibitors, as set forth above, formulated into compositions together with one or more physiologically tolerable or acceptable diluents, carriers, adjuvants or vehicles that are collectively referred to herein as carriers. Compositions suitable for such contact or administration can comprise physiologically acceptable aqueous or nonaqueous solutions, dispersions, suspensions or emulsions, whether or not sterile. The resulting compositions can be, in conjunction with the various methods described herein, for administration or contact with a human ornithine δ-aminotransferase. Whether or not in conjunction with a pharmaceutical composition, “contacting” means that a human ornithine δ-aminotransferase and one or more inhibitor compounds are brought together for purpose of binding and/or complexing such an inhibitor compound to the enzyme. Amounts of a compound effective to inhibit a human ornithine δ-aminotransferase may be determined empirically, and making such determinations is within the skill in the art. Inhibition or otherwise affecting a human ornithine δ-aminotransferase activity includes reduction, mitigation and/or modulation, as well as elimination of OAT activity, glutamate production, glutamine synthesis, cell proliferation and/or tumor growth.

It is understood by those skilled in the art that dosage amount will vary with the activity of a particular inhibitor compound, disease state, route of administration, duration of treatment, and like factors well-known in the medical and pharmaceutical arts. In general, a suitable dose will be an amount which is the lowest dose effective to produce a therapeutic or prophylactic effect. If desired, an effective dose of such a compound, pharmaceutically-acceptable salt thereof, or related composition may be administered in two or more sub-doses, administered separately over an appropriate period of time.

Methods of preparing pharmaceutical formulations or compositions include the step of bringing an inhibitor compound into association with a carrier and, optionally, one or more additional adjuvants or ingredients. For example, standard pharmaceutical formulation techniques can be employed, such as those described in Remington's Pharmaceutical Sciences, Mack Publishing Company, Easton, PA.

Regardless of composition or formulation, those skilled in the art will recognize various avenues for medicament administration, together with corresponding factors and parameters to be considered in rendering such a medicament suitable for administration. Accordingly, with respect to one or more non-limiting embodiments, the disclosed compounds may be utilized as inhibitor compounds for the manufacture of a medicament for therapeutic use in the treatment or prevention of a disease or disorder associated with hOAT activity, expression, or overexpression. Suitable diseases or disorders may include cell proliferative diseases or disorders, which may include but are not limited to hepatocellular carcinoma (HCC) and non-small cell lung cancer (NSCLC).

Generally, with respect to various embodiments, the disclosed subject matter can be directed to method(s) for the treatment of a pathologic proliferative disorder. As used herein, the term “disorder” refers to a condition in which there is a disturbance of normal functioning. A “disease” is any abnormal condition of the body or mind that causes discomfort, dysfunction, or distress to the person affected or those in contact with the person. Sometimes the term is used broadly to include injuries, disabilities, syndromes, symptoms, deviant behaviors, and atypical variations of structure and function, while in other contexts these may be considered distinguishable categories. It should be noted that the terms “disease”, “disorder”, “condition” and “illness”, are equally used herein.

According to certain embodiments, the disclosed methods can be specifically applicable for the treatment of malignant proliferative disorders, including malignant proliferative disorders that express human ornithine δ-aminotransferase (hOAT). As used herein, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. Accordingly, the compounds, compositions, and methods disclosed herein may be used in the treatment of non-solid and solid tumors.

Malignancy, as contemplated herein, may be selected from the group consisting of melanomas, carcinomas, leukemias, lymphomas and sarcomas, which express hOAT. Malignancies that can be treated by the methods disclosed herein, including malignancies that express OAT can comprise but are not limited to hematological malignancies (including leukemia, lymphoma and myeloproliferative disorders), hypoplastic and aplastic anemia (both virally induced and idiopathic), myelodysplastic syndromes, all types of paraneoplastic syndromes (both immune mediated and idiopathic) and solid tumors (including bladder, rectum, stomach, cervix, ovarian, renal, lung, liver, breast, colon, prostate, GI tract, pancreas and Karposi). More particularly, according to certain embodiments, the compounds and compositions used in conjunction can be used in methods for the treatment or inhibition of non-solid cancers, e.g. hematopoietic malignancies such as all types of leukemia, e.g. acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma, as well as for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of Vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma, retinoblastoma, carcinoma of the lacrimal gland, sarcoma of the orbit, brain, spinal cord, vascular system, hemangiosarcoma and Kaposi's sarcoma.

The compounds and compositions disclosed berein may be administered in methods of treatment as known in the art. Accordingly, various such compounds and compositions can be administered in conjunction with such a method in any suitable way. For example, administration may comprise oral, intravenous, intraarterial, intramuscular, subcutaneous, intraperitoneal, parenteral, transdermal, intravaginal, intranasal, mucosal, sublingual, topical, rectal or subcutaneous administration, or any combination thereof.

According to some embodiments, the treated subject may be a mammalian subject. Although the methods disclosed herein are particularly intended for the treatment of proliferative disorders in humans, other mammals are included. By way of non-limiting examples, mammalian subjects include monkeys, equines, cattle, canines, felines, mice, rats and pigs.

The terms “treat, treating, treatment” as used herein and in the claims mean ameliorating one or more clinical indicia of disease activity in a subject having a pathologic disorder. “Treatment” refers to therapeutic treatment. Those in need of treatment are mammalian subjects suffering from any pathologic disorder. By “patient” or “subject in need” is meant any mammal for which administration of a compound or any pharmaceutical composition of the sort described herein is desired, in order to prevent, overcome, modulate or slow down such infliction. To provide a “preventive treatment” or “prophylactic treatment” is acting in a protective manner, to defend against or prevent something, especially a condition or disease.

More generally, the disclosed methods may be directed to affecting, modulate, reducing, inhibiting and/or preventing the initiation, progression and/or metastasis (e.g., from the liver elsewhere or to the liver from any other organ or tissue) of a malignant pathologic proliferative disorder associated with OAT activity. (See, e.g., Lucero O M, Dawson D W, Moon R T, et al. A re-evaluation of the “oncogenic” nature of Wnt/beta-catenin signaling in melanoma and other cancers. Curr Oncol Rep 2010, 12, 314-318; Liu Wei; Le Anne; Hancock Chad; Lane Andrew N; Dang Chi V; Fan Teresa W-M; Phang James M. Reprogramming of proline and glutamine metabolism contributes to the proliferative and metabolic responses regulated by oncogenic transcription factor c-MYC. Proc. Natl. Acad. Sei. USA 2012, 109(23), 8983-8988; and Tong, Xuemei; Zhao, Fangping; Thompson, Craig B. The molecular determinants of de novo nucleotide biosynthesis in cancer cells. Curr. Opin. Genet. Devel. 2009, 19(1), 32-37.)

### ILLUSTRATIVE EMBODIMENTS

The following Embodiments are illustrative and should not be interpreted to limit the scope of the claimed subject matter.

Embodiment 1. A compound of the following formula or a dissociated form, zwitterion form, or a salt thereof:

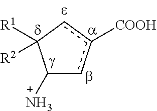

wherein a double bond is present between the α and ε carbons or between the α and β carbons, and wherein each of R¹ and R² is independently selected from a halogen such as F, Cl, Br, and I.

Embodiment 2. The compound of embodiment 1 in zwitterion form comprising an ammonium moiety and a carboxylate moiety.

Embodiment 3. The compound of embodiment 1, wherein the double bond is between the α and ε carbons.

Embodiment 4. The compound of embodiment 1, wherein the double bond is between the α and β carbons.

Embodiment 5. The compound of embodiment 1, wherein at least one of RI and R² is F.

Embodiment 6. The compound of embodiment 5, wherein the compound is a salt comprising a substituent selected from an ammonium substituent, a carboxylate substituent, and a combination thereof.

Embodiment 7. The compound of embodiment 6, wherein the ammonium salt has a counter ion that is the conjugate base of a protic acid.

Embodiment 8. The compound of embodiment 1 in a pharmaceutical composition comprising a pharmaceutically-acceptable carrier component.

Embodiment 9. The compound of embodiment 1 of a formula:

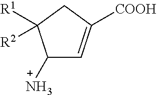

wherein at least one of R¹ and R² is F.

Embodiment 10. The compound of embodiment 9, wherein each of R¹ and R² is F.

Embodiment 11. The compound of embodiment 1 of a formula:

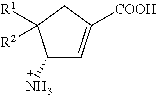

Embodiment 12. The compound of embodiment 1 of a formula:

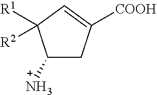

Embodiment 13. The compound of embodiment 9, wherein the compound is a salt comprising a substituent selected from an ammonium substituent, a carboxylate substituent, and a combination thereof.

Embodiment 14. The compound of embodiment 13, wherein the ammonium salt has a counter ion that is the conjugate base of a protic acid.

Embodiment 15. The compound of embodiment 9 in a pharmaceutical composition comprising a pharmaceutically-acceptable carrier component.

Embodiment 16. The compound of embodiment 11 or 12, wherein the compound is a salt comprising a substituent selected from an ammonium substituent, a carboxylate substituent, and a combination thereof.

Embodiment 17. The compound of embodiment 16, wherein the ammonium salt has a counter ion that is the conjugate base of a protic acid.

Embodiment 18. The compound of embodiment 11 or 12 in a pharmaceutical composition comprising a pharmaceutically-acceptable carrier component.

Embodiment 19. A pharmaceutical composition comprising: (i) the compound of embodiment 1; and (ii) a pharmaceutically suitable carrier, diluent, or excipient.

Embodiment 20. A pharmaceutical composition comprising: (i) the compound of embodiment 9; and (ii) a pharmaceutically suitable carrier, diluent, or excipient.

Embodiment 21. A pharmaceutical composition comprising: (i) the compound of embodiment 11 or 12; and (ii) a pharmaceutically suitable carrier, diluent, or excipient.

Embodiment 22. A method of modulating human ornithine δ-aminotransferase (hOAT) activity, the method comprising contacting the compound of embodiment 1 with a medium comprising OAT, wherein the compound is present in an amount sufficient to modulate hOAT activity.

Embodiment 23. The method of embodiment 22, wherein the double bond is between the α and ε carbons.

Embodiment 24. The method of embodiment 22, wherein the double bond is between the α and β carbons.

Embodiment 25. The method of embodiment 22, wherein at least one of R¹ and R² is F.

Embodiment 26 The method of embodiment 22, wherein the compound is a salt comprising a substituent selected from an ammonium substituent, a carboxylate substituent, and a combination thereof.

Embodiment 27. The method of embodiment 26, wherein the ammonium salt has a counter ion that is the conjugate base of a protic acid.

Embodiment 28. The method of embodiment 22, wherein the contact is in vivo.

Embodiment 29. A method of reducing activity of an hOAT expressed by a human cancer, the method comprising contacting the compound of embodiment 1 with the cancer expressing an hOAT, wherein the compound is present in an amount that is effective to reduce hOAT activity.

Embodiment 30. The method of embodiment 29, wherein the double bond is

between the α and ε carbons.

Embodiment 31. The method of embodiment 29, wherein the double bond is

between the α and β carbons.

Embodiment 32. The method of embodiment 29, wherein at least one of R¹ and R²is F.

Embodiment 33. The method of embodiment 32, wherein the compound is provided in a pharmaceutical composition.

Embodiment 34. The method of embodiment 33, wherein the contact is in vivo.

Embodiment 35. The method of embodiment 34, wherein the contact is with a human subject in need thereof.

Embodiment 36. A method for treating cancer in a subject in need thereof, the method comprising administering to the subject a therapeutically effective amount of the compound of embodiment 1.

Embodiment 37. The method of embodiment 36, wherein the double bond is between the α and ε carbons.

Embodiment 38. The method of embodiment 36, wherein the double bond is between the α and β carbons.

Embodiment 39. The method of embodiment 36, wherein at least one of R¹ and R² is F.

Embodiment 40. The method of embodiment 36, wherein the compound is a compound of a formula

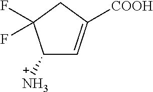 [SMILES: [NH3+][C@H]1C=C(C(=O)O)CC1(F)F]

Embodiment 41. The method of embodiment 36, wherein the compound is a compound of a formula

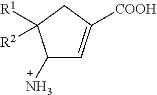

wherein at least one of R¹ and R² is F, or a salt thereof.

Embodiment 42. The method of embodiment 36, wherein each of R¹ and R² is F.

Embodiment 43. The method of embodiment 36, wherein the cancer is characterized by expression or overexpression of human ornithine δ-aminotransferase (hOAT).

Embodiment 44. The method of embodiment 36, wherein the cancer is hepatocellular carcinoma (HCC).

Embodiment 45. The method of embodiment 36, wherein the cancer is non-small cell lung cancer (NSCLC).

### EXAMPLES

The following Examples are illustrative and should not be interpreted to limit the scope of the claimed subject matter The following non-limiting Examples and data illustrate various aspects and features relating to the disclosed compounds, compositions, and methods including the treatment of diseases and disorders associated with hOAT activity, expression, or overexpression, and/or reduction of human ornithine aminotransferase activity, such as cell proliferative diseases and disorders including, but not limited to hepatocellular carcinoma (HCC) and non-small cell lung cancer (NSCLC). While the utility of this invention is illustrated through the use of several compounds and compositions which can be used therewith, it will be understood by those skilled in the art that comparable results are obtainable with various other compound(s), as are commensurate with the scope of this invention.

### Example 1

### Title—Discovery and Mechanistic Studies of SS-1-148, a Selective Mechanism-Based Inactivator of Human Ornithine Aminotransferase

### Introduction

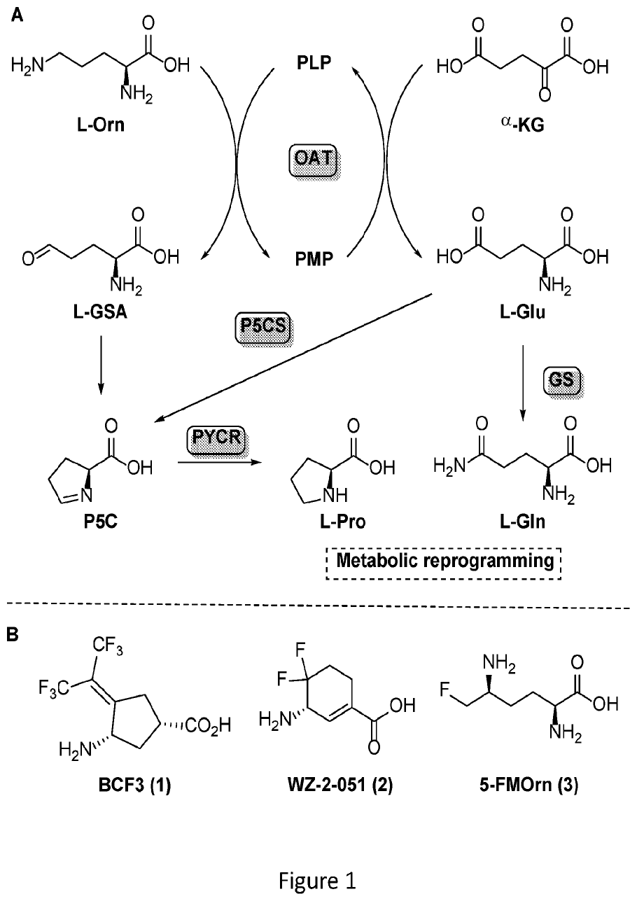

Human ornithine δ-aminotransferase (hOAT; EC2.6.1.13) is a pyridoxal-5′-phosphate (PLP)-dependent enzyme that catalyzes two coupled transamination reactions, converting L-ornithine (L-Orn) to afford L-glutamate-γ-semialdehyde (L-GSA) in the first half-reaction with the generation of L-glutamate (L-Glu) from α-ketoglutarate (α-KG) in the second half-reaction (FIG. 1A).¹ The afforded intermediate L-GSA is a spontaneous equilibrium species of Δ1-pyrroline-5-carboxylate (P5C), while P5S can, in turn, afford L-proline catalyzed by P5C reductases (PYCRs).² On the other hand, the generated L-Glu can also be converted to P5S by pyrroline-5-carboxylate synthase (P5CS), thus participating in proline metabolism (FIG. 1A).² Proline biosynthesis was identified as the most substantially altered amino acid metabolism in human tumor tissues of hepatocellular carcinoma (HCC), featured by accelerated proline consumption, hydroxyproline accumulation, and correlation with increased α-fetoprotein (AFP) levels and poor prognosis in HCC.³ In addition, glutamine synthetase (GS) catalyzes L-Glu's conversion to L-glutamine (L-Gln).⁴ L-Gln is highly required by cancer cells to support anabolic processes, thus promoting cellular proliferation.

HCC is the predominant liver malignancy and ranks among the most common leading causes of cancer-associated mortality worldwide. Our previous work identified the OAT gene as one of the seven overexpressed genes in the spontaneous HCC-developing livers from Psammomys obesus (sand rat) identified by DNA microarray analysis.⁵ Moreover, the treatment (0.1 and 1.0 mg/kg: PO) of selective hOAT mechanism-based inactivator (MBI) BCF3 (1) remarkably decreased the serum AFP levels and inhibited tumor growth in an HCC mouse model,⁵ underscoring the antitumor effects of pharmacological selective hOAT inhibition. MBI is a type of molecule that initially acts as an alternative substrate of the target enzyme and is then transformed into active species that can further inactivate the enzyme through specific covalent modification, tight-binding electrostatic interaction, or any other functionally irreversible inhibition.⁶⁻⁷ MBIs are typically unreactive before the initial bonding with the active site of the target enzyme, thereby usually exhibiting significant target specificity and selectivity.⁸ Overall, hOAT can be considered as a potential therapeutic target for HCC, and selectively inactivating hOAT may provide a novel opportunity to discover effective HCC treatment.

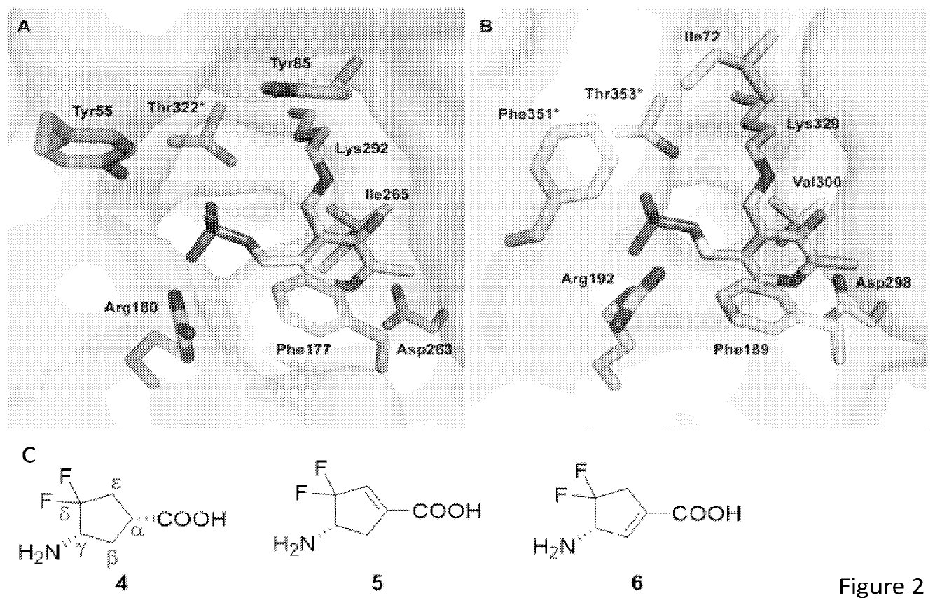

However, the major challenge for discovering selective MBI of hOAT is to overcome the irreversible inhibition on other aminotransferases,⁶ especially γ-aminobutyric acid aminotransferase (GABA-AT) that has a high structural similarity with hOAT.¹ There are only two significant differences in the active site pocket of their homodimer structures: Tyr85 and Tyr55 in hOAT are replaced by Ile72 and Phe351* in GABA-AT, respectively (FIG. 2).¹ Moreover, Ile72 and Phe351* are responsible for the slightly narrower but more hydrophobic active site of GABA-AT relative to hOAT. In contrast, the hydroxyl group of Tyr55 serves as a hydrogen bond acceptor to interact with the charged C-2 amino group of substrates, while Tyr85 is a significant determinant of substrate specificity and conformationally flexible to adopt bulky substrates.¹

According to the high similarity between these two aminotransferases, a preliminary screening against hOAT was carried out previously using our stock GABA-AT inhibitors.⁵ A cyclopentane-based analog, termed BCF3 (1, FIG. 1B), bearing bis(trifluoromethyl) group as its warhead, was identified to be a selective MBI of hOAT while only showing millimolar reversible inhibition on GABA-AT. The recent mechanistic studies have revealed that one of its trifluoromethyl groups occurs fluoride ion elimination, leading the ligand to covalently modify the catalytic Lys292 through the conjugate addition (Scheme 1A).⁹⁻¹⁰ It is considered that the steric bulky bis(trifluoromethyl) group may not be easy to access the relatively narrower pocket of GABA-AT, influencing the initial binding pose between the ligand and the enzyme, which may be responsible for its reversible inhibition on GABA-AT. BCF3 has been demonstrated to be effective in vivo as abovementioned⁵ and is being investigated in extensive IND-labelling toxicity assessment and efficacy studies in HCC patient-derived xenografted (PDX) models. Cyclohexene-based analog WZ-2-051 (2, FIG. 1B) bears an enlarged ring system and a difluoro group.¹¹ WZ-2-051 exhibited a 23-fold improvement in inactivation efficiency (defined by the k_{inact}/K₁ ratio) against hOAT compared to BCF3 while showing 13.3-fold selectivity over GABA-AT. The subsequent mechanistic studies have disclosed that WZ-2-051 undergoes two-step fluoride ion elimination and finally inactivates hOAT through an addition-aromatization mechanism (Scheme 1B).¹¹ An additional example of selective hOAT inactivator is 5-FMOrn (3) inspired by the structure of hOAT substrate L-Orn and the structure of non-selective GABA-AT inactivator AFPA,¹² inactivates hOAT via an enamine pathway by forming a ternary adduct (Scheme 1C).¹³

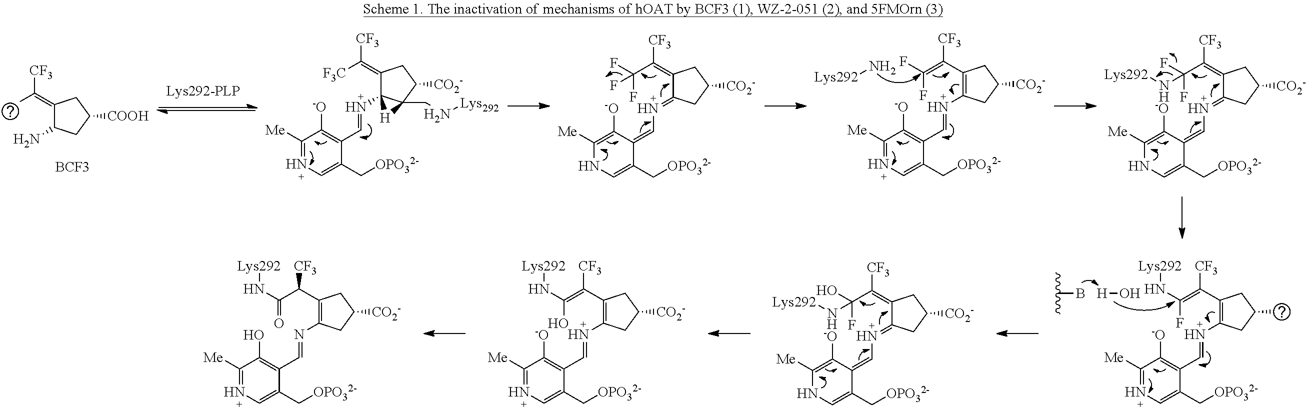

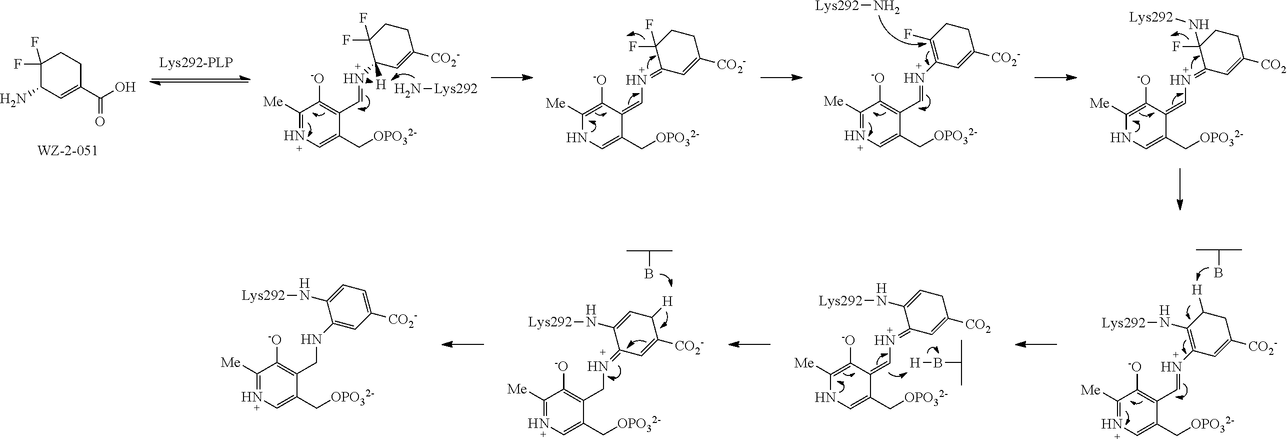

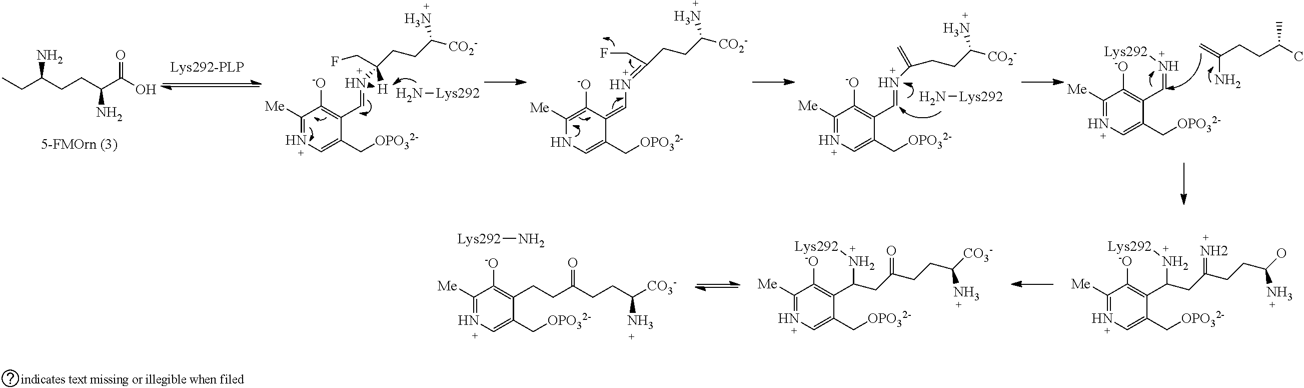

It should be noted that the α-amino group of 5-FMOrn forms a strong hydrogen bond with the phenol group of Tyr55 in the hOAT crystal complex (PDB entry 20AT).¹⁴ Moreover, it was also observed that hydrogen bonds between their carboxylate groups and Tyr55 in the hOAT crystal complexes with BCF3 (PDB entry 6OIA) and WZ-2-051 (PDB entry 6V8C),^{9,11} thereby demonstrating that the interaction with Tyr55 plays a significant in ligand specificity and selectivity. However, among the published hOAT inactivators, only BCF3 demonstrates potent irreversible inhibition on hOAT, weak reversible inhibition on GABA-AT (Kᵢ=4.2 mM), and no inhibition on aspartate aminotransferase (Asp-AT) and alanine aminotransferase (Ala-AT) up to 4 mM,⁵ leading to promising hOAT selectivity. Therefore, discovering novel selective MBIs of hOAT will facilitate the investigation of hOAT inactivators as potential therapeutic approaches for HCC.

In 2000, (1R,4S)-4-amino-3,3-difluorocyclopentanecarboxylic acid (4, FIG. 2C) was found to be a reversible inhibitor against GABA-AT (kᵢ=0.19 mM).¹⁵ Fifteen years later, it was further demonstrated to be an hOAT inactivator.⁵ However, compound 4 exhibits high binding affinity (K₁=7.8 mM) towards hOAT and a low maximum rate of inactivation (K_{inact}=0.02 min⁻¹) against hOAT, eventually causing a modest inactivation efficiency (k_{inact}/k₁=0.003 min⁻¹mM⁻¹), which limits us to elucidate its inactivation and turnover mechanisms.

The present technology provides for novel cyclopentene-based analogs, including SS-1-148 (6) by incorporating an additional double bond into its cyclopentane ring system of 4, which are demonstrated to be a highly potent and selective hOAT inactivator. Mechanistic studies utilizing crystallization, protein and molecular mass spectrometry, transient state measurements, and computational simulations to reveal a novel non-covalent inactivation mechanism of SS-1-148.

### RESULTS AND DISCUSSION

Synthesis of Cyclopentene Analogs 5 and 6 Bearing Difluoro Group. Incorporating a double bond has been demonstrated to be an effective strategy for improving inactivation efficiency, which influences the configuration of the initial external aldimine and the adjunct proton's acidity due to the α,β-unsaturated carboxylate.¹⁶⁻¹⁷ Therefore, cyclopentene-based analogs 5 and 6 bearing difluoro group were designed based on the structure of the parent compound 4 (FIG. 2C), intending to apply this approach to the development of more potent hOAT inactivators.

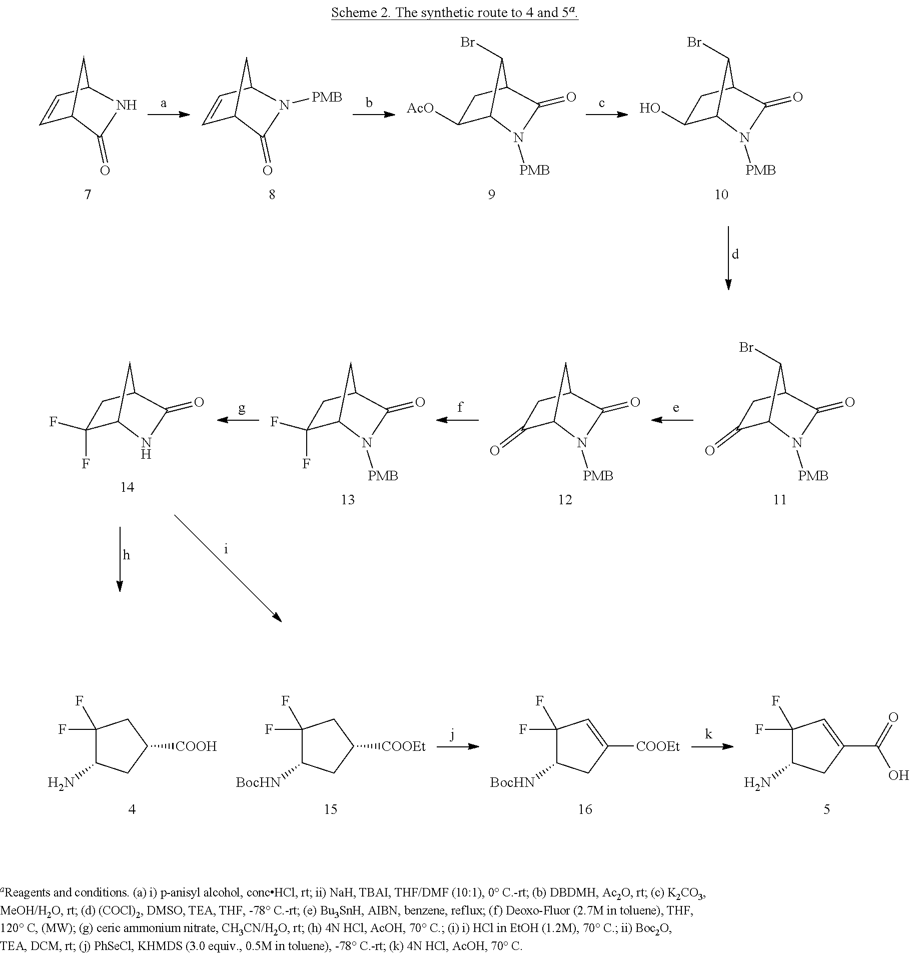

The synthetic route to prepare compound 5 initiated from the (1R)-(−)-2-azabicyclo[2.2.1hept-5-en-3-one(7; Vince lactam¹⁸; CAS#: 79200-56-9) to afford the key bicyclic intermediate 9 according to the procedure we developed previously.¹⁵ Acetyl group of 9 was then converted to a hydroxyl group under acidic conditions followed by Swern oxidation, yielding ketone intermediate 11. After that, dehydrobromination undertook to replace the bromo group on the bridgehead of 11 with hydrogen to form 12 under Bu₃SnH/AIBN conditions based on the procedure in the literature.¹⁵ Intermediate 12 further reacted with Deoxo-Fluor reagent under microwave conditions to afford difluoro intermediate 13. And then, 13 underwent PMB deprotection using ceric ammonium nitrate (CAM), and the afforded lactam 14 was treated with HCl/EtOH to yield the ethyl ester cyclopentane under reflux conditions. The introduction of the Boc protective group was followed to obtain intermediate 15. Interestingly, when it was attempted to prepare an intermediate of 15 bearing a selenenyl group at the α-position of its carboxylate group of 15 under the KHMDS (3.0 equiv.) conditions for the follow-up α-elimination reaction,¹⁶ intermediate 16 bearing an α, ε-conjugated carboxylate group was afforded directly as the sole product. 1D and 2D NMR were performed to validate the structure of 16. In the end, the new cyclopentene-based analog 5 was afforded accordingly after deprotection under acidic conditions. The parent cyclopentane-based analog 4 was also prepared from intermediate 14 that easily undertook one-step acidic hydrolysis and was evaluated together with new analogs and BCF3 in the subsequent kinetic studies.

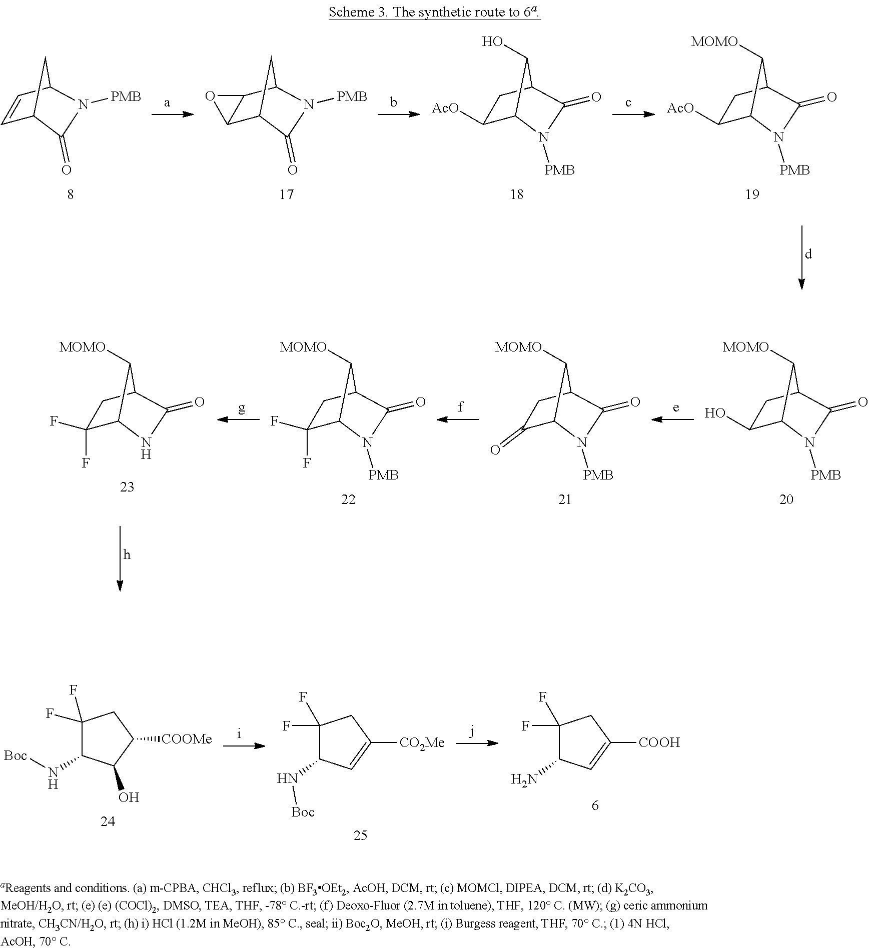

The synthetic route to prepare 6 started from the synthesis of the critical bicyclic intermediate 20 from PMB-protected Vince lactam 8 following the efficient procedure we recently published.¹⁷ The hydroxyl group of 20 was then converted into ketone intermediate 21 by Swern oxidation. Difluoro intermediate 22 was obtained through the same fluorination conditions described above. Furthermore, the PMB group of 22 was removed under CAM conditions followed by lactam hydrolysis and Boc protection that obtained the cyclopentane intermediate 24. The hydroxyl group at the β-position of the methyl carboxylate group of 24 was dehydrated using Burgess reagent¹⁹⁻²⁰ under reflux conditions, forming cyclopentene intermediate 25 containing an α, β-conjugated carboxylate group. The final product 6 was afforded accordingly following the deprotection conditions as above.

Kinetic Studies of analogs 4-6. The kinetic results in Table 1 indicate that all three difluoro-based compounds 4-6 exhibited irreversible inhibition on hOAT but reversible inhibition on GABA-AT, demonstrating that they are selective hOAT inactivators. Moreover, in comparison with 4 and 5 that displayed high millimolar binding affinity (K₁) of hOAT, while 6 exhibited a significant improvement (K₁=0.06 mM). The partition ratio determination and fluoride ion release results (Table 1) revealed that the majority of 4 and 5 participate in the alternative turnover pathway rather than inactivation path while releasing large amounts of fluoride ion, resulting in their high binding affinities. On the other hand, 6 also showed the highest rate constant (k_{inact}) relative to 4 and 5, thereby leading to superior inactivation efficiency (k_{inact}/K₁) of 6 relative to the parent compound 4 (1.333 vs. 0.003 min⁻¹mM⁻¹; 444-fold improvement). In addition, it was also noted that the inhibitory activities of cyclopentene-based 5 and 6 against GABA-AT are about 10-times weaker than cyclopentane-based 4, suggesting improved selectivity over GABA-AT. Compound 6, named SS-1-148, exhibited comparable inactivation efficiency against hOAT while retaining reversible inhibition on GABA-AT relative to the preclinical stage BCF3. SS-1-148 also did not show noticeable inhibition on Asp-AT and Ala-AT up to 10 mM. Therefore, elucidating the potential inactivation and turnover mechanisms of SS-1-148 in hOAT is of interest.

TABLE 1
Kinetic Constants for the Inactivation of hOAT and Reversible Inhibition of GABA-AT by 4-6^{a}

| | | hOAT | | | | | GABA-AT |
|---|---|---|---|---|---|---|---|
| | | K_{inact} | K_{I} | K_{inact} /K_{I} | Partition | Fluoride | Kᵢ |
| Cmpd | Structure | (min⁻¹) | (mM) | (min⁻¹ mM⁻¹) | ratio | ion release^{c} | (mM) |
| 4 | 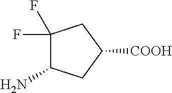 [SMILES: N[C@H]1C[C@@H](C(=O)O)CC1(F)F] | 0.01 ± 0.00 | 3.96 ± 1.27 | 0.003 | 2257 | 3395± 31 | 0.09 |
| 5 | 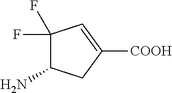 [SMILES: N[C@H]1CC(C(=O)O)=CC1(F)F] | 0.03 ± 0.01 | 2.13 ± 0.90 | 0.014 | 817 | 754 ± 57 | 1.34 |
| SS-1-148 (6) | 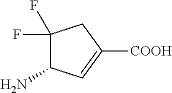 [SMILES: N[C@H]1C=C(C(=O)O)CC1(F)F] | 0.08 ± 0.01 | 0.06 ± 0.03 | 1.333 | 33.9 | 33.9 ± 0.8 | 1.06 |
| BCF3 (1) | 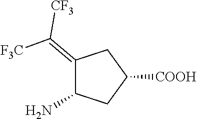 [SMILES: N[C@H]1C[C@@H](C(=O)O)CC1=C(C(F)(F)F)C(F)(F)F] | 0.09 ± 0.01 | 0.09 ± 0.03 | 1.00 | 12^{b} | 79^{b} | 5.16 |

^{a}K_{inact} and K_{I} values were determined by the equation: K_{obs} = k_{inact} *[I]/(K_{I} + [I]) and presented as means and standard errors. Kᵢ was calculated by the equation: Kᵢ = IC₅₀/(1 + [S]/Kₘ). IC₅₀ values were obtained using nonlinear regression of a 9-point enzymatic assay with a 2-fold serial dilution against GABA-AT. The partition ratios were determined under conditions in the presence of α-KG, while fluoride ion release results were determined under conditions without α-KG. ^{b}Data were extracted from Reference 9. ^{c}The fluoride ion release experiments were performed in triplicate. Data presented as mean with standard deviation.

Proposed Inactivation Mechanism Pathways of SS-1-148 (6). Based on previous mechanistic studies of other related GABA-AT/hOAT inactivators,^{7, 11} three possible pathways are initially proposed and summarized in Scheme 4. At the beginning of inactivation, SS-1-148 would capture the PLP moiety from internal aldimine Lys292-PLP, forming external aldimine M1. After that, M1 would undertake deprotonation to form a quinonoid species M2 followed by a fluoride ion elimination step afforded the shared intermediate M3 for three following pathways. Pathway a is proposed based on recent findings on cyclohexene-based analog WZ-2-051.11 The electrophilic C_{δ} position of intermediate M3 could be attacked by Lys292, forming a covalent bond (M4) through a conjugate addition. The quinonoid species M4 would occur the second fluoride ion elimination to afford the final adduct M5 (Scheme 4). Pathway b is inspired by the inactivation mechanisms of CPP-115 and OV329 in GABA-AT, which is achieved through a water-mediated mechanism to afford tight electrostatic interactions between their carboxylates and arginine residues in the active site.^{16, 21} A water molecular catalyzed by Lys292 would attack the electrophilic C_{δ} position of intermediate M3 followed by another fluoride ion elimination step that would afford enol/carbonyl group (M7) rather than establishing a covalent bond with the residue in hOAT. Pathway c is proposed according to the typical enamine mechanism.²² Lys292 would attack the C_{4′} position of the aldimine instead of the C_{δ} position while releasing enamine intermediate M9, which would reattack the imine linkage of the internal aldimine (PLP-Lys292) and produce a covalent adduct M10.

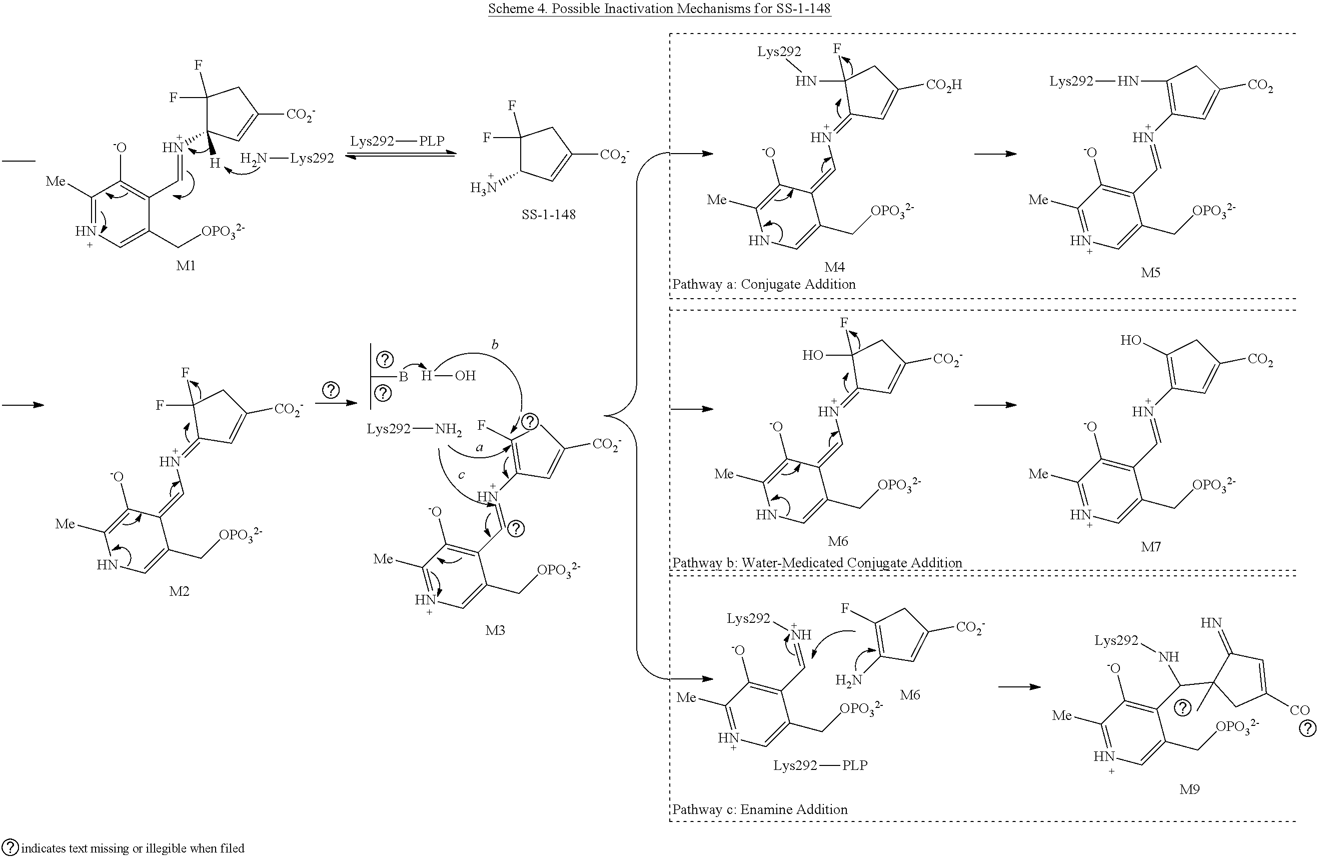

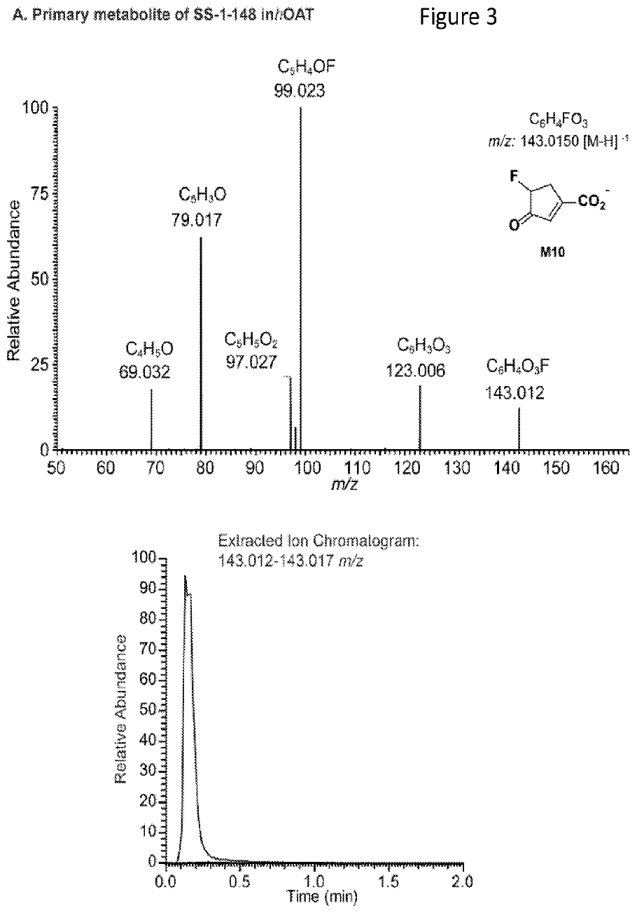

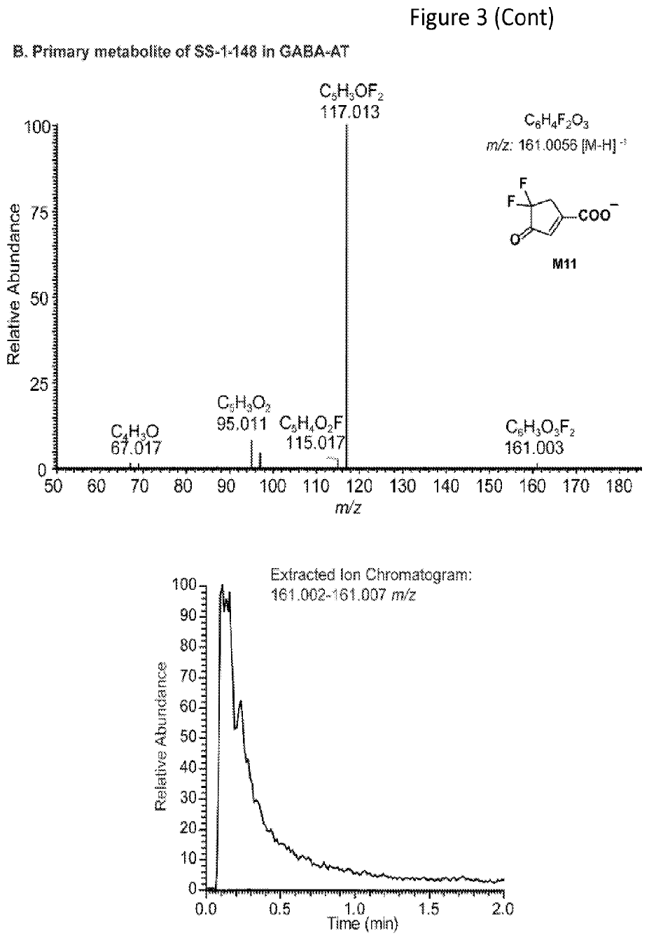

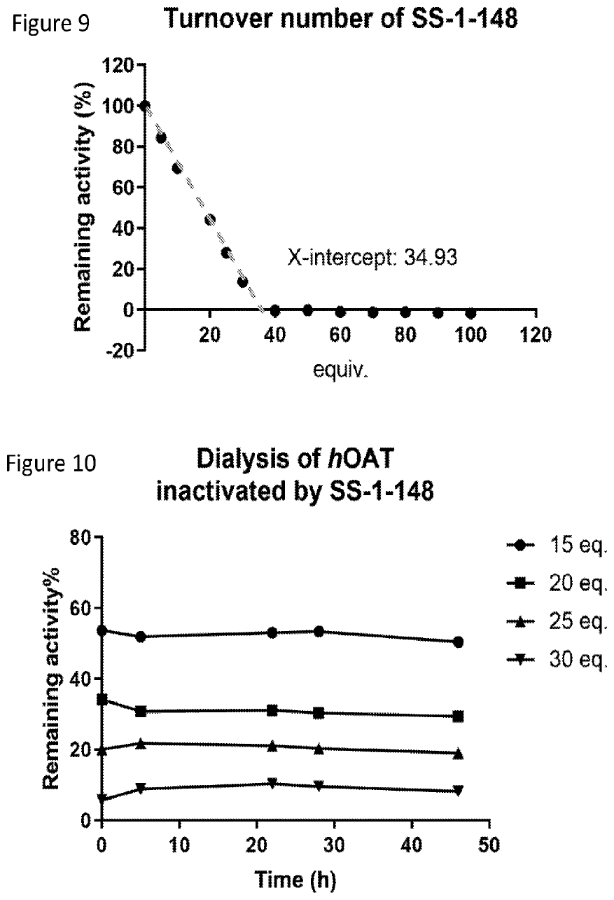

Plausible Turnover Mechanisms of SS-1-148 with hOAT and GABA-AT. SS-1-148 exhibited a relatively high partition ratio (33.9-fold, Table 1 and FIG. 9), indicating 34.9 equivalents of SS-1-148 are turned over per active site for each equivalent of the compound, leading to inactivation. Moreover, 33.9±0.8 equivalents of fluoride ion (Table 1) are released per inactivation event, suggesting that the primary turnover pathway only occurs one fluoride ion elimination step. Previously, electrostatic potential (ESP) charge calculations were carried out to demonstrate that the fluorine atom apparently decreases the nucleophilicity of enamine intermediate (similar to the structure of M8),¹¹ which may prevent the occurrence of enamine addition. Metabolomics study of SS-1-148 in hOAT (FIG. 3A) showed that the molecular weight and fragmentations of the primary metabolite match the structure of M10 in Scheme 5 that is the corresponding hydrolyzed product from enamine intermediate M8. We thus considered that Pathway c in Scheme 4 might be the major turnover pathway of SS-1-148 in hOAT. On the other hand, the primary metabolite of SS-1-148 in GABA-AT was identified. The molecular weight and fragmentations shown in FIG. 3B suggest that SS-1-148 acts as a substrate to yield ketone M11 bearing difluoro group (Scheme 5).

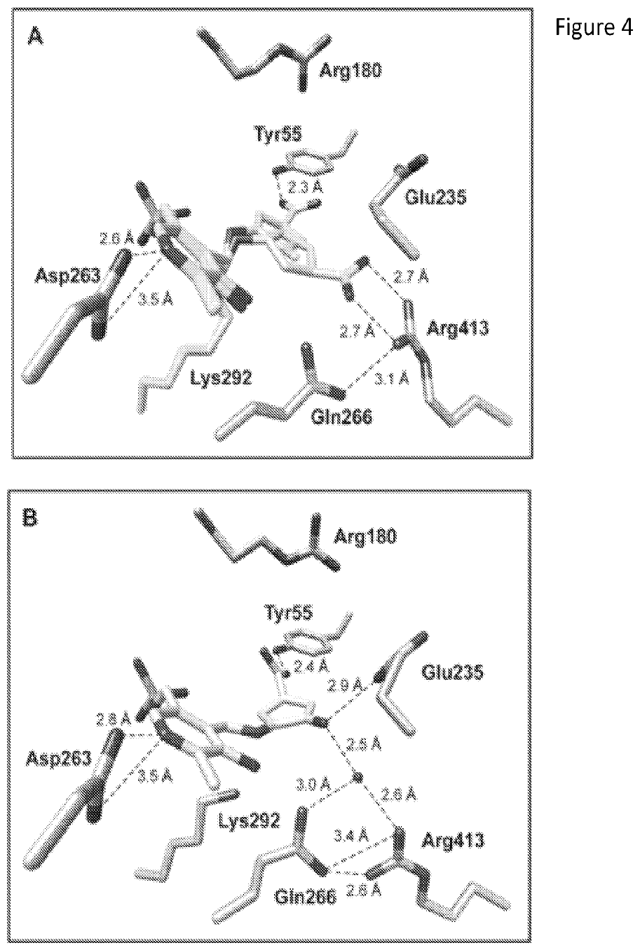

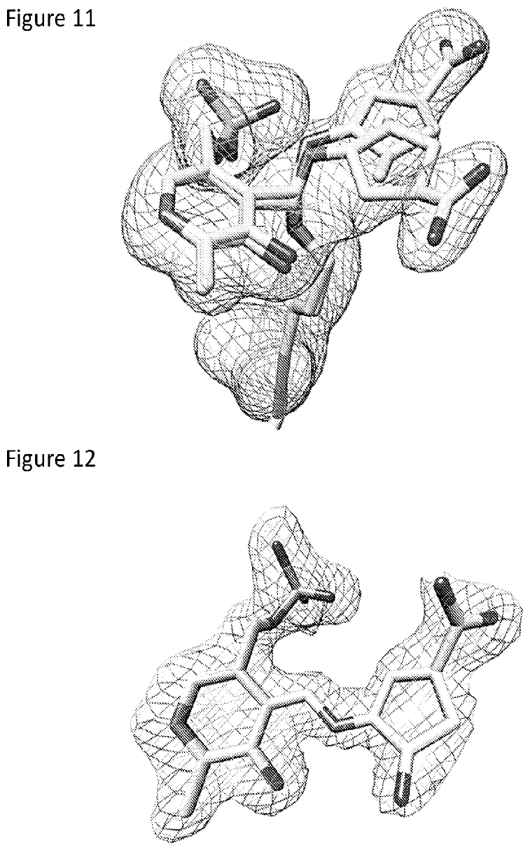

In an attempt to capture the primary intermediate of the non-inactivation pathway, hOAT holoenzyme crystals were utilized to perform one-hour soaking experiments with SS-1-148 solution. The hOAT structure was solved by molecular replacement from a previously reported structure (PDB entry 1OAT). The space group for the SS-1-148 soaking structure was found to be P 3₂ 2 1, and the structure contains three copies of the protein monomer in one asymmetric unit. The crystal structure (PDB entry 7LK1) shown in FIG. 4A and FIG. 11 indicates that PLP was covalently linked to SS-1-148, while a covalent bond between Lys292 and SS-1-148 was revealed in the soaked crystal. The covalent bond between Lys292 and SS-1-148 represents a stable gem-diamine species that has never been observed in any previous hOAT/ligand crystals.^{9-11, 23} This observation further validated the gem-diamine precursors (M12 or M13, Scheme 5) of enamine intermediate M8. Moreover, two structural conformations of this intermediate were observed in soaked crystal in which differ in the position of the carboxylate group of SS-1-148. The first conformation forms a hydrogen bond between Tyr55 and the carboxylate of SS-1-148, while the second conformation forms a salt bridge with Arg413. The conclusion about two alternative conformations for the intermediate structure was based on the positive density in proximity to Arg413 and Tyr55 as well as the relatively high B-factors for any single conformation by itself. An alternative explanation could include two different, yet structurally similar, intermediate species that could interact with the protein active site in different ways.

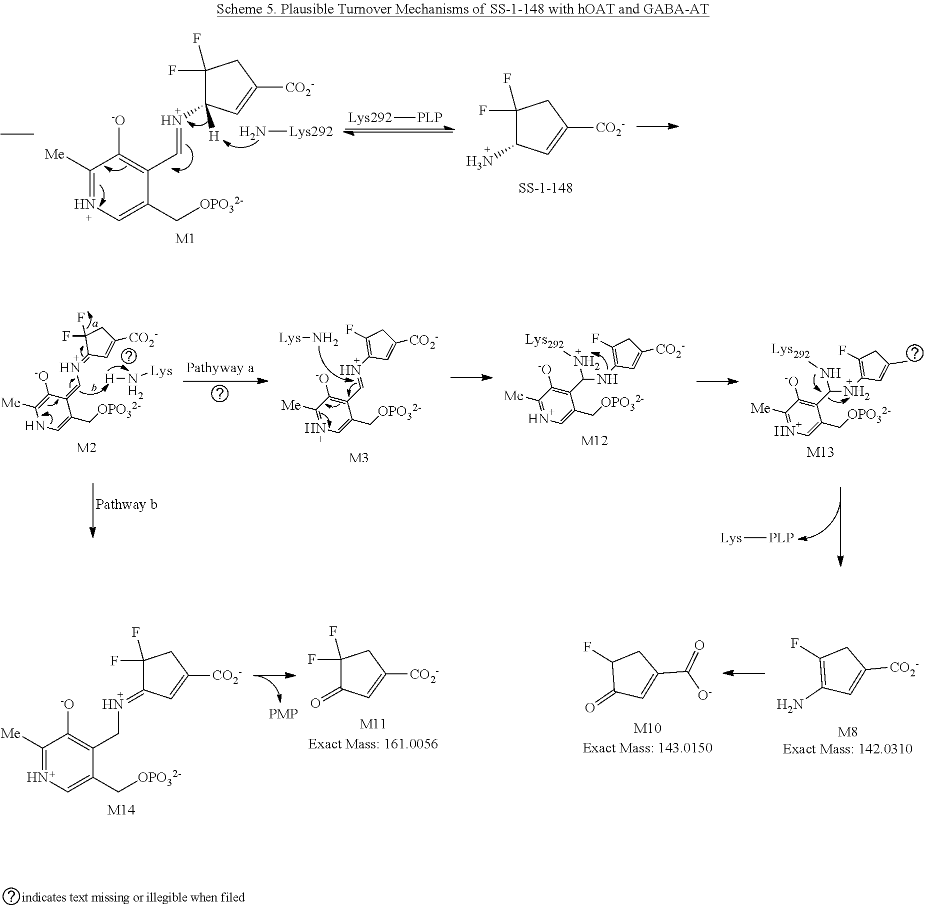

Overall, according to the evidence, the major turnover mechanisms for SS-1-148 in hOAT and GABA-AT are proposed in Scheme 5, respectively. After capturing the PLP ligand from Lys292, M1 undergoes deprotonation, and the afforded quinonoid M2 occurs a fluoride ion elimination (Pathway a; Scheme 5) in hOAT to afford aldimine intermediate M3 bearing monofluorine. The majority of M3 is attacked by Lys292 at its C_{4′} position, forming the first gem-diamine M12, followed by proton transfer²⁴⁻²⁵ that affords the second gem-diamine M13 that is further converted to enamine metabolite M8 and the internal aldimine. In the end, M8 hydrolyzes to ketone M10 as the primary metabolite of SS-1-148 in hOAT. In contrast, quinonoid intermediate M2 only undergoes electron transfer to yield ketimine M14 hydrolyzes to release PMP and M11 as the primary metabolite in GABA-AT. The behavior of SS-1-148 in GABA-AT mimics a transamination reaction without eliminating any fluoride ion, consistent with its reversible inhibition on GABA-AT. It should be noted that given by the structural differences between hOAT and GABA-AT, the known selective hOAT inactivators (1-3) described above contain bulky moiety or α-amino group to improve the ligand specificity and selectivity over GABA-AT. However, the selectivity of SS-1-148 is associated with its competitive relationship with substrate GABA.

Plausible Inactivation Mechanisms of SS-1-148 with hOAT. The irreversibility of the inhibition of hOAT by SS-1-148 was evaluated by a time-dependent dialysis experiment up to 48 h against a buffer consisting of excessive PLP and α-KG (FIG. 10). The remaining activity of hOAT was unchanged, demonstrating that SS-1-148 is an hOAT irreversible inhibitor. To reveal the structure of the final product in the inactivation reaction, we co-crystallized hOAT with an excessive amount of SS-1-148. The crystal structure (PDB entry 7LK0) was solved following the same procedure described above, and the space group for SS-1-148 cocrystal (FIG. 4B and FIG. 12) was found to be P 3₁ 1 2. It also contains three copies of the protein monomer in one asymmetric unit. Similar to the soaking crystal structure shown in FIG. 4A, PLP is covalently linked to SS-1-148 moiety in the cocrystal structure. However, no covalent bond is noticed between Lys292 and the final product. Moreover, in the published crystal structure of the native enzyme (holo-hOAT), Arg413 typically forms a salt bridge with Glu235, which is found intact for several cocrystals of hOAT with different inactivators.^{14, 23, 26} In the hOAT/SS-1-148 soaking result (FIG. 4A), the salt bridge of Arg413-Glu235 is broken due to the existence of an alternative salt bridge between the carboxylate of one intermediate and Arg413. Interestingly, the Arg413-Glu235 salt bridge is also found to be broken in the hOAT/SS-1-148 cocrystal, while no direct interaction is observed between Arg413 and the ligand. Instead, Arg413, Gln266, and the final product are hydrogen-bonded to the same water molecule. Additionally, the oxygen group of the ligand shows a hydrogen bond with Glu235 in a distance of 2.9 Å (FIG. 4B), which may contribute to stabilizing the ligand in the hOAT pocket. The ligand structure in hOAT/SS-1-148 cocrystal indicates that the inactivation may be attributed to Pathway B in Scheme 4, generating a final product close to the structure of M7 (theoretical mass: 370.06 Da).

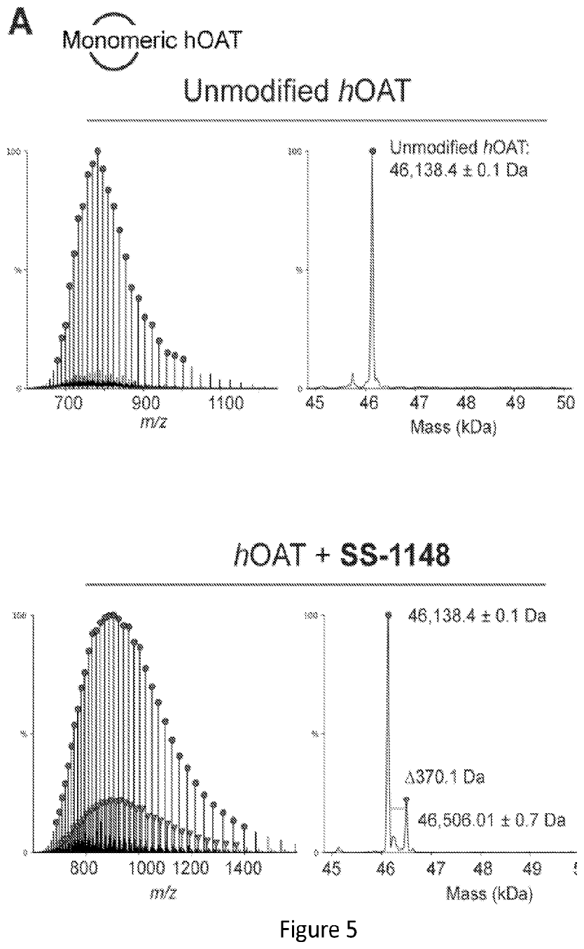

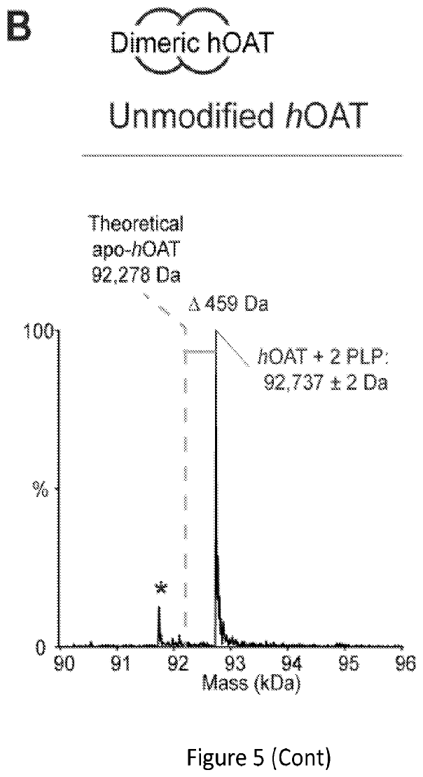

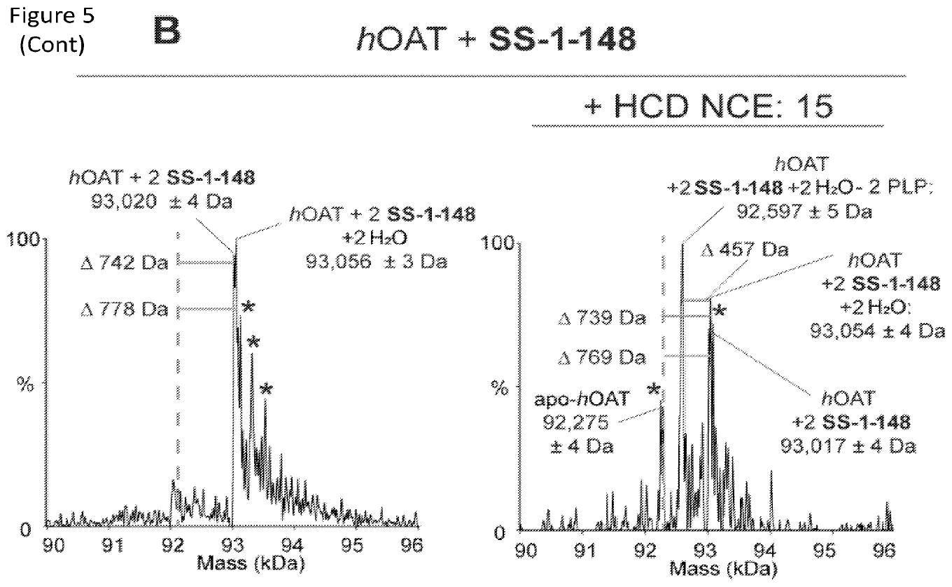

Intact protein mass spectrometry (intact MS) has been previously applied to elucidate inactivation mechanisms of hOAT inactivators.^{9, 11} Here, through denaturing, intact LC-MS (FIG. 5A, right) experiments using hOAT sample fully inactivated by SS-1-148, only ˜16% of hOAT was found to be covalently modified, leading to a mass shift of 370.01 Da. The majority of hOAT remained unmodified (FIG. 5A, top) in comparison with native hOAT as a control (FIG. 5A, bottom). The adduct mass (370.01 Da) was found to be identical with the theoretical mass of gem-diamine M15 (370.06 Da; Scheme 6) that could be in equilibrium with the non-covalent form M7. However, one would expect a higher abundance of covalently adducted enzyme under these conditions where the enzyme is fully inactivated. This finding thus supported a major non-covalent, inactivation mechansim between hOAT and SS-1-148.

Native protein mass spectrometry (native MS) was further employed to identify solution-state, non-covalent protein interactions that are preserved in the gas phase and characterize the binding of the SS-1-148 product in hOAT. The results shown in FIG. 5B indicate that unmodified hOAT appears as one dimeric form (92,737±2 Da) that is 459 Da more massive than the theoretical apo-hOAT dimer. This mass shift is consistent with two PLP-bound internal aldimines in the two active sites of the dimer (459 Da shift observed; 460 Da theoretical). A proteolytic form of unomdified hOAT (FIG. 5B, Unmodified hOAT (*)) was also observed, but only under control conditions. In contrast, native, dimeric hOAT sample fully inactivated by SS-1-148 was observed with two high abundace masses of 93,020±4 Da and 93,056±3 Da, corresponding to a mass shift of 742 Da and 778 Da, respectivley (FIG. 5B, hOAT+SS-1-1-148 (left)). Several more massive species that can be attributed to salt adducts were observed (FIG. 5B, hOAT+SS-1-1-148 (left) (*)). No unmodified, apo-hOAT or PLP-bound hOAT was observed. The observed 93,020±4 Da mass is consistant with the theoretical mass of M7 (370.06 Da) in both active sites (theoretical: 93,018 Da). Moreover, the 93,056±3 Da observed mass is consistent with M7 and an active-site water in both protein chains (theoretical: 93,056 Da), validating the final product and the key water molecule observed in the cocrystal structure (FIG. 4B).

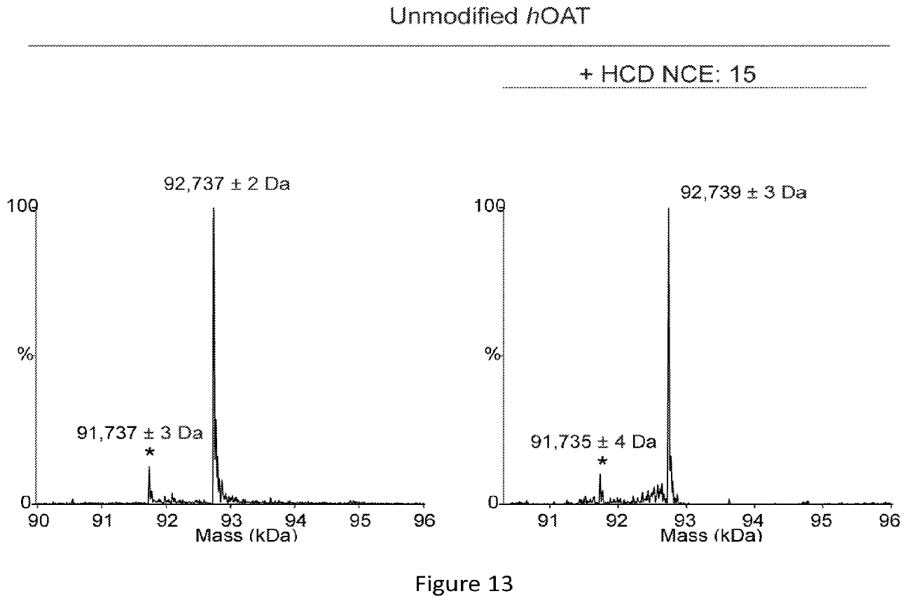

To further probe the SS-1-148-hOAT interaction by mass spectrometry, higher-energy collisional dissocation (HCD) was applied to unmodified and, separately, treated hOAT to dissociate protein: ligand interactions and eject ligands from the enzyme complex. Under sub-proteolytic dissociative conditions (HCD NCE:15), no mass defect was observed for unmodified hOAT (FIG. 13). However, under these same dissociative conditions, two additional masses were produced for SS-1148-inactivated hOAT (FIG. 5B, hOAT+SS-1-1-148 (right)). One mass, consistent with apo-hOAT, was observed at 45% relative abundance (observed: 92,275±4 Da; theoretical: 92,278 Da). A second mass of 92,597±5 Da was observed at 100% relative abundance. Compared to the previously observed dimer mass that is consistent with each active sites bound by M7 and one water molecule, this species has a 457 Da mass defect. Intestestingly, the high abunance mass shift cannot be explained by loss of a single M7±H₂O adduct from the protein dimer (theoretical mass for M7-hOAT: 92,648 Da; theoretical mass for one M7-hOAT±H₂O: 92,666 Da). However, the mass defect observed through HCD activation can be explained by the loss of PLP from both active sites, while the active-site waters and SS-1-148 moieties are retained (observed: 457 Da; theoretical: 460 Da). This finding is surprising, given that the same collsional energy does not eject PLP from the unmodified protein dimer. However, based on the cocrystal structure identified binding of SS-1-148 and water to Tyr55, Glu235, Gln266 and Arg413, it is likely that protein stability is increased upon SS-1-148 binding. and in the gas phase, breaking these interactions is unfavorable relative to cleavage of PLP.

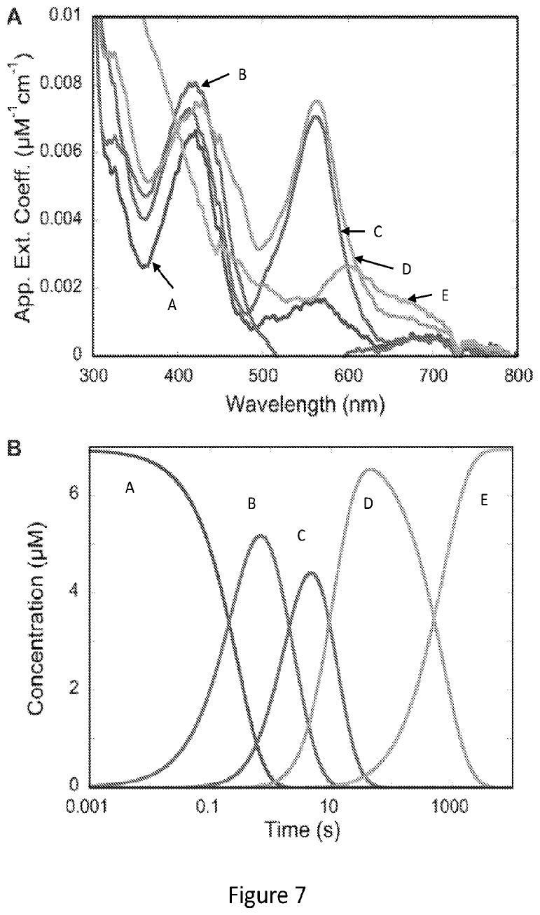

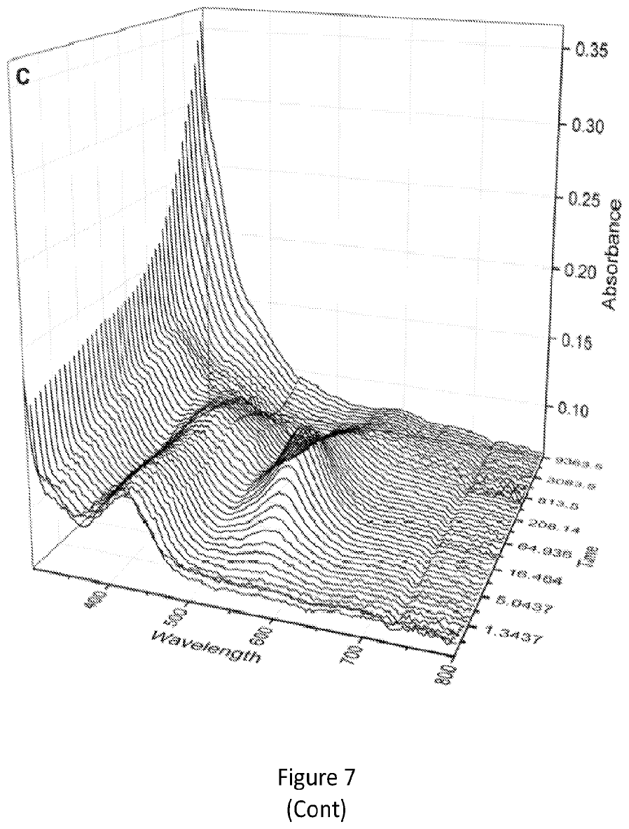

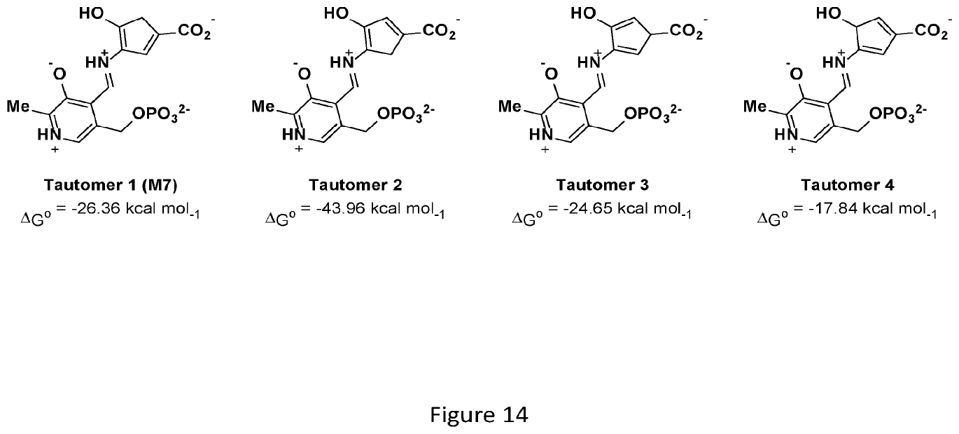

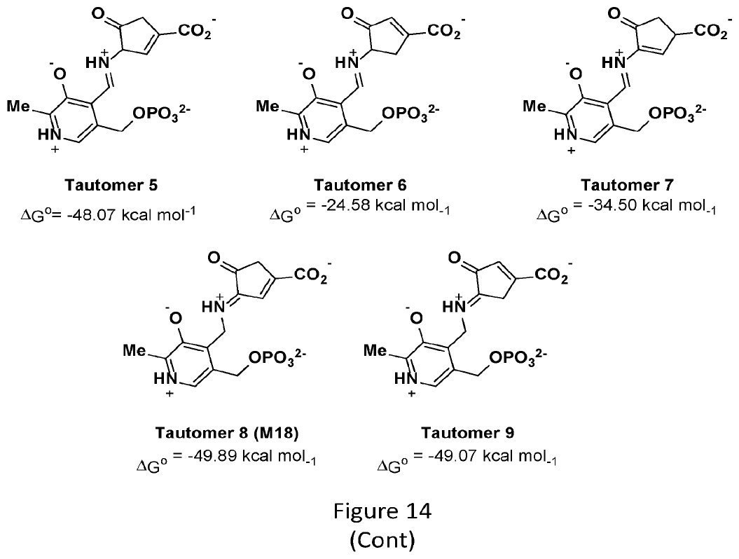

As no apo-enzyme observed by native MS, and approximately 84% of hOAT was in an apo-enzyme state under denaturing conditions, the results demonstrate that non-covalent M7 is the primary form after inactivation, while covalently-bonded M15 is the minor form that is in equilibrium with M7. Therefore, M7 generated from the water-mediated Pathway b (Scheme 4) seems to be the final product of SS-1-148 after inactivation. However, given its highly conjugated structure, M7 is easy to occur tautomerization. Gibb's free energy calculation²⁸ was thus performed on MOPAC to assess the stabilities of M7 and related tautomeric forms in the hOAT's active site. The results shown in FIG. 14 suggest that, in comparison with the enol form M7 (ΔG^{o}=−26.36 kcal mol⁻¹), its corresponding ketone (tautomer 5, ΔG^{o}=−48.07 kcal mol⁻¹) and ketimine (tautomer 8, ΔG^{o}=−49.89 kcal mol⁻¹) are relatively more stable in the active site. Moreover, it should be noted that a species that shows absorbance at ˜275 nm was determined as the final product in the subsequent transient state measurements (FIG. 7C), thereby ruling out all external aldimines (e.g., M7 and tautomer 5) that absorbances are appropriately at 420 nm as the final form of SS-1-148. On the other hand, the results above indicate that the final state is more like a gem-diamine or ketimine, while their typical absorbances appear at about 330-340 nm.^{24, 29} According to the literature, in comparison with the ketoenamine moiety containing protonated aldimine and deprotonated hydroxyl, the enolimine with the neutral states of the hydroxyl and aldimine group exhibits significantly lower absorption maximum (410 nm vs. 330 nm).³⁰ Taken together, both gem-diamine M15 in equilibrium with M7 and ketimine M18 tautomerized from M7 may undergo an extra step of electron transfer to form M16 and M19, respectively. Due to the neutral states of gem-diamine M16 and ketimine M19, their absorption maximums are able to drop from 330 nm to 275 nm, thereby matching the absorbance of the final species observed in the transient state measurements.

Overall, the inactivation pathways of SS-1-148 in hOAT are summarized in Scheme 6. The initial external aldimine M1 occurs deprotonation catalyzed by Lys292 and forms the first quinonoid M2. A fluoride ion elimination is followed to yield monofluoro aldimine M3 from M2. The C_{4′} position of the majority of M3 (˜97%; determined by partition ratio) is attacked by Lys292 while releasing an enamine metabolite that hydrolyzes to ketone M10 as the primary metabolite (Pathway a; Scheme 5 and 6). The C_{δ} position of a small portion of M3 (˜3%) goes through a water-mediated nucleophilic attack (Pathway b; Scheme 6), generating the second quinonoid M6. Intermediate M6 further undergoes another fluoride ion elimination to form M7. After that, a small fraction of M7 (˜16%) may covalently bind to Lys292 at its C_{4′} position through a gem-diamine form (M15) (Pathway c; Scheme 6). The generated gem-diamine M15 can be in equilibrium with M7, while it can also occur further electron transfer to generate a neutral gem-diamine form 16 as a more stable state. On the other hand, the majority of M7 (˜84%) tautomerizes to a more favorable ketimine M18, which is followed by additional electron transfer that affords ketimine M19 as the primary final product (Pathway d; Scheme 6).

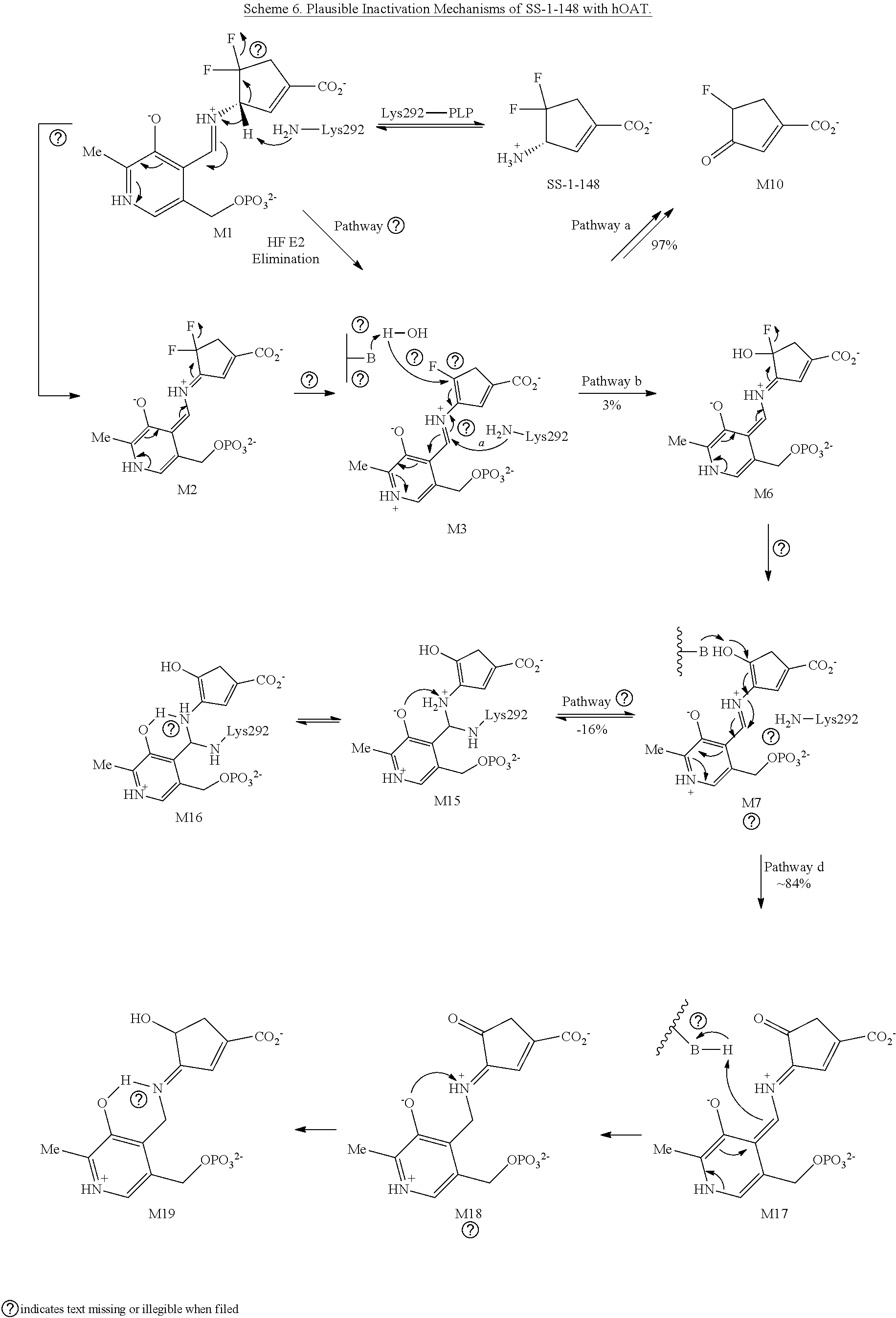

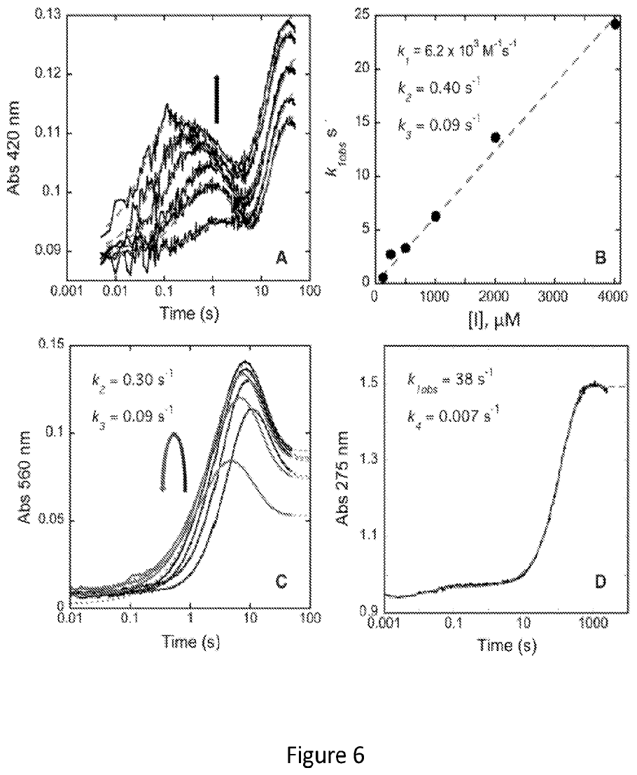

Transient State Measurements of hOAT Inhibited by SS-1-148 As a variety of transient states are involved in the proposed mechanisms, we subsequently performed rapid mixing spectrophotometric measurements in an attempt to capture the kinetics of the inhibition of hOAT by SS-1-148. This approach takes advantage of the conjugated species that accumulate sequentially in PLP-dependent transaminase reactions.¹⁰ The inhibition reaction that occurs with SS-1-148 was interpreted in combination with X-ray structures abovementioned acquired for different stages of the reaction progression (vide infra). The experimental data shown in FIGS. 6 and 7 indicate that the reaction of SS-1-148 with hOAT is complex Four discernable phases were observed with evidence of at least two parallel reaction paths (FIG. 6), indicating that the mechanistic conclusions drawn are necessarily from undetermined models Within the deadtime of the stopped-flow instrument, the reaction of SS-1-148 with hOAT formed a spectrum (˜420 nm) consistent with an external aldimine (FIGS. 7A-B(A)). Titration of hOAT with SS-1-148 modulated the rate and extent of accumulation of a second external aldimine contribution that is presumably additive with the aldimine formed in the deadtime (FIGS. 7A-B(B)). The second external aldimine forms with a rate dependence that indicates a bimolecular reaction (6.2×10³ M⁻¹s⁻¹) suggest that SS-1-148 combines with hOAT in the least two ways, resulting in parallel reaction paths (FIGS. 6A-B). The intensity of the combined external aldimine spectra decays partially with the accumulation of a spectral transition characteristic for a quinonoid species (˜560 nm) at 0.4 s⁻¹ (FIGS. 6A-B and 7A-B). The apparent quinonoid species is formed with concomitant and partial decay of the external aldimine transitions that are approximately equal in amplitude to that gained with the second external aldimine accumulation. This suggests that these species lie on the same reaction path.

It has been reported that the deprotonation of the initial external aldimine MI and stepwise fluoride elimination steps proposed in Scheme 6 are typically considered as an EICB mechanism elimination.³¹⁻³² The electron-withdrawing effect of fluorine and the protonated nitrogen of the aldimine may stabilize the formed carbanion state during the elimination reaction.³²⁻³³ Meanwhile, quinonoid transient state M2 is supposed to be formed between the first and second external aldimines (M1 and M3). However, the stopped-flow experimental results more suggest that antiperiplanar hydrogen and fluorine in M1 undergoes an unusual Lys292-assisted E2 mechanism³¹ with simultaneous loss of a proton, the release of fluoride ion, and formation of alkene as a more favorable fluoride elimination pathway, affording the second external aldimine M3 as the single transient state (Pathway e; Scheme 6). This result has never been observed experimentally in previous mechanistic studies of other related PLP-dependent aminotransferase inactivators.^{1, 7}

Assuming a quinonoid extinction coefficient of ˜30 mM⁻¹cm⁻¹, the fractional accumulation of the quinonoid observed is ˜20% of total reacting species at 1 mM SS-1-148. The quinonoid then decays at a rate of 0.09 s⁻¹, while the residual transitions assigned to external aldimine species broaden and persist (FIGS. 7A-B(D)). Quinonoid M6 seems to be the first quinonoid species (˜560 nm) that can be observed based on the turnover (Pathway a; Scheme 5) and inactivation (Pathway b; Scheme 6) mechanisms. The final phase observed occurs with a rate constant of 0.007 s⁻¹. And in this phase, the features of the external aldimine decay with a pronounced increase in absorption intensity at ˜275 nm, indicative of a loss of conjugation (FIGS. 7A-B(E)), consistent with gem-diamine M16 and ketimine M19 proposed as the final products in Scheme 6.

Collectively, these data support a dominant pathway comprised of multiple distinct external aldimine species that ultimately decay to a less conjugated product (turnover mechanism; Pathways a and e; Scheme 6) and a second minor pathway that forms an initial external aldimine more slowly but then proceeds through a quinonoid intermediate and decays to also form a non-conjugated product (inactivation mechanism; Pathways b-e; Scheme 6). The data shown in FIG. 7C indicate that the proportion of each pathway is dependent on the concentration of SS-1-148. Higher concentrations of SS-1-148 diminish the accumulation of the quinonoid species but do not alter the observed rates of accumulation and decay, suggesting that the more rapid and dominant pathway sequesters a larger fraction of enzyme at higher SS-1-148 concentrations.

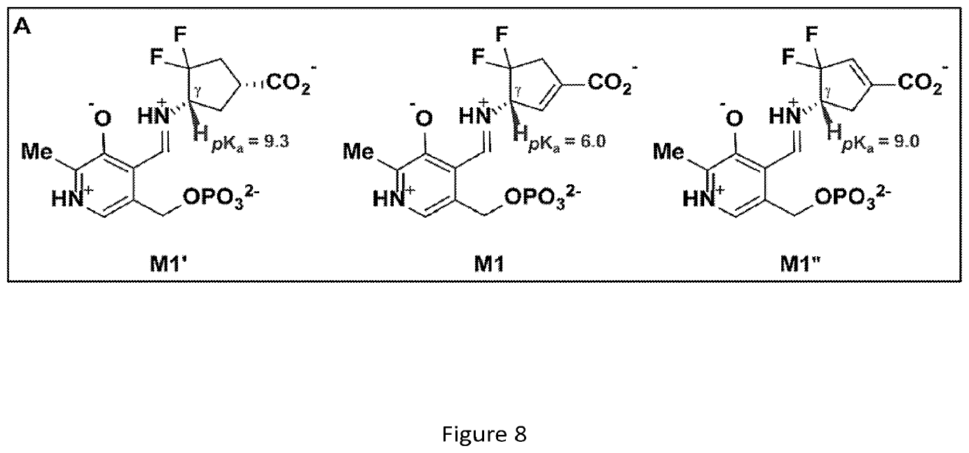

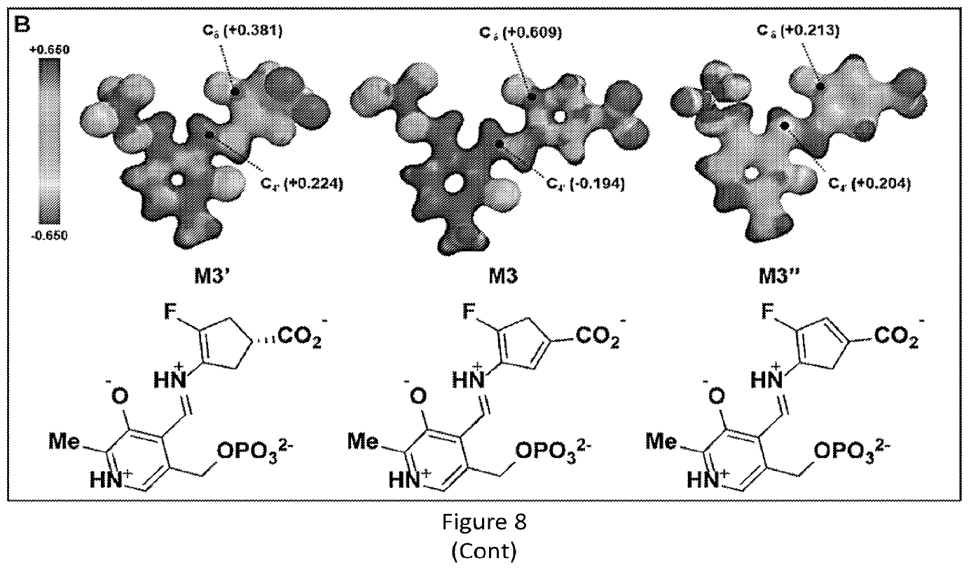

The Significance of the Conjugated Alkene of SS-1-148. After better understanding the inactivation and turnover mechanisms of SS-1-148 (6), computational calculations were carried out to compare analogs 4-6. Our previous studies have revealed that incorporating an extra double bond into the cyclopentane ring system establishes an α, β-conjugated carboxylate and facilitates the deprotonation step due to the increased acidity of the adjunct proton, leading to the enhanced rate constant.¹⁶⁻¹⁷ Theoretical pKₐ calculations were performed using the DFT/B3LYP method³⁴ at 298K to predict the proton's acidity at the C_{γ} position of difluoro analogs 4-6 (FIG. 2C). The results shown in FIG. 8A suggest that the C_{γ} hydrogen of PLP-bound SS-1-148 (M1) with an α, β-conjugated carboxylate system displays the lowest pKₐ value among the three analogs, while the pKₐ value of the corresponding proton in PLP-bound 5 (M1″) is not noticeably affected by the introduced double bond at the C_{α} and C_{ε} positions relative to the parent cyclopentane 4 (M1′). Taken together with their distinct k_{inact} values (Table 1), these findings may suggest that the more acidic hydrogen at the C_{γ} position facilitates the first external aldimine of SS-1-148 (M1) to initiate an E2 fluoride ion elimination step rather than the typical E1CB elimination reaction, thus contributing to its improved rate constant.

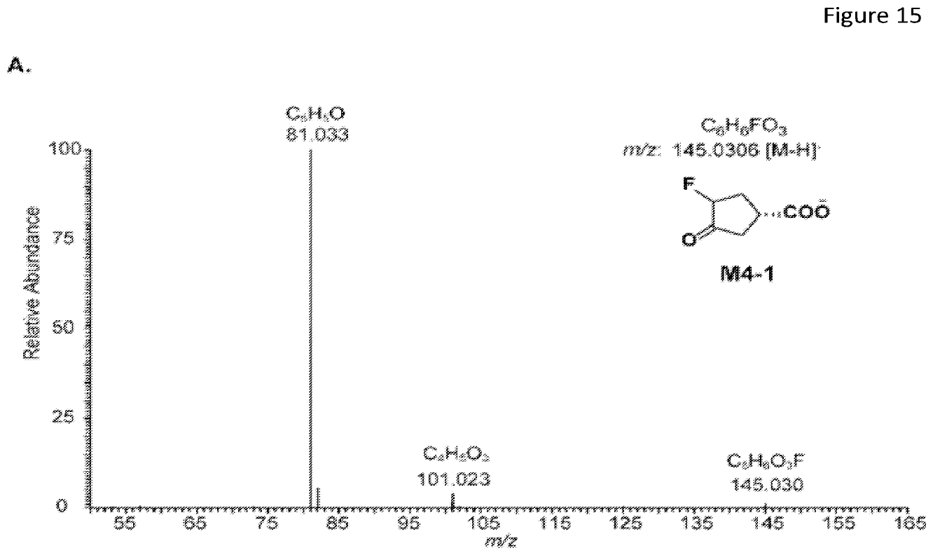

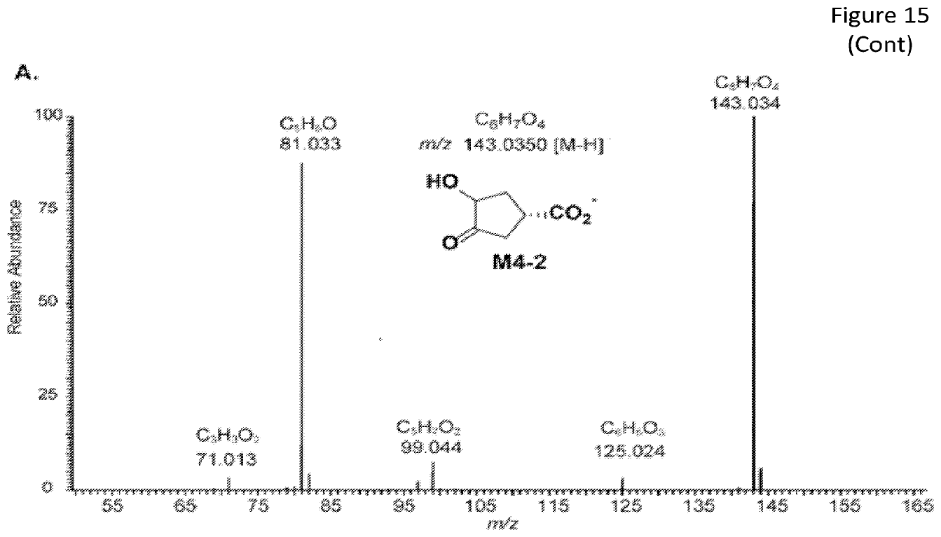

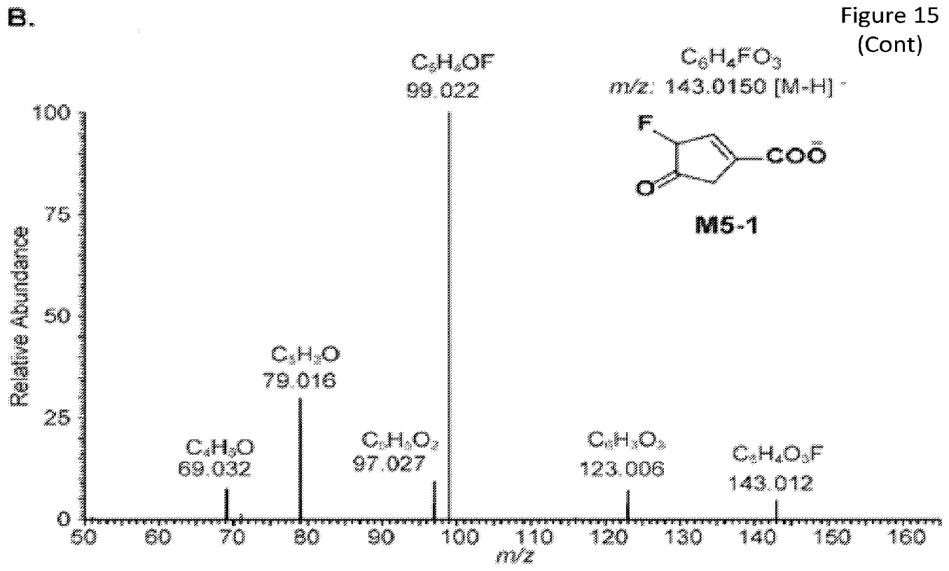

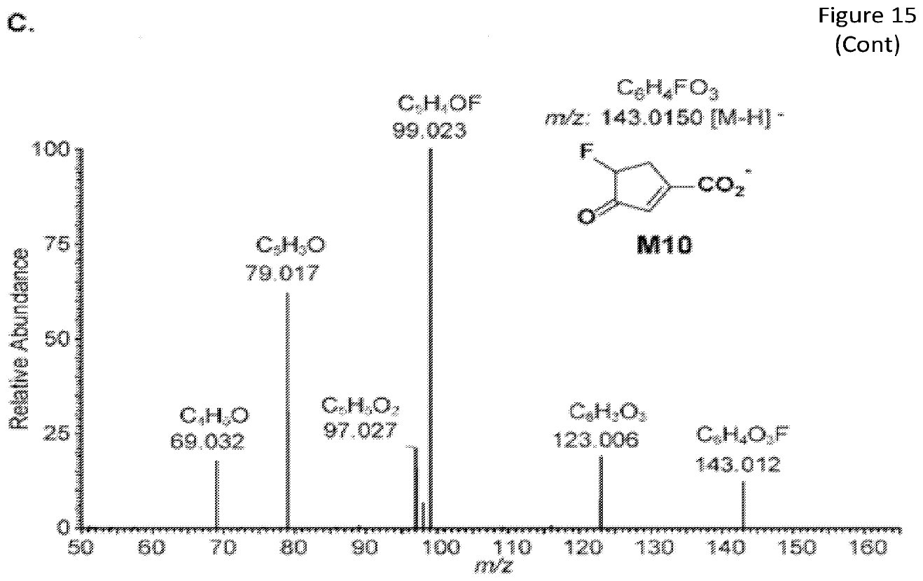

The partition ratio determination and fluoride ion release results (Table 1) elucidated that 4 and 5 predominantly participate in the alternative turnover pathway rather than the inactivation pathway while releasing large amounts of fluoride ions. The electron density maps and ESP charge calculations¹¹ (FIG. 8B) exhibit that the electrophilicity of C_{δ} in the transient state M3 of SS-1-148 (6) is much higher than that in the corresponding transient states of 4 and 5 (M3′ for 4 and M3″ for 5), indicating that the Co position of M3 is more reactive than that in the other two intermediates. On the other hand, the C_{4′} positions in the aldimine linkage of M3′ and M3″ displays comparable electrophilicity that is much greater than the result of M3, which demonstrates M3′ and M3″ are easier to be attacked by catalytic Lys292 to trigger their turnover pathways, eventually resulting in significantly high partition ratios of 4 and 5. Furthermore, additional metabolomics studies in hOAT revealed that, similar to SS-1-148 in hOAT, cyclopentene analog 5 generated a ketone bearing single fluorine as its primary metabolite (M5-1. FIG. 15B). In contrast, cyclopentane 4 not only formed a monofluorinated ketone (M4-1, FIG. 15A) but also generated a ketone metabolite bearing a hydroxyl group (M4-2, FIG. 15A) that was not detected in the metabolomics results of cyclopentene analogs. According to the proposed mechanisms of SS-1-148 in Scheme 6, we thus consider that the incorporated double bond also plays a critical role in stabilizing the conjugated system.

### CONCLUSION

Previously, (S)-3-amino-4,4-difluorocyclohex-1-enecarboxylic acid (WZ-2-051, 2) inactivates hOAT through a covalent addition-aromatization mechanism (Scheme 1B). However, it still shows apparent irresponsible inhibition on GABA-AT. (S)-3-amino-4,4-difluorocyclopent-1-enecarboxylic acid (SS-1-148, 6) was identified to exhibit comparable inactivation efficiency with preclinical stage selective hOAT inactivator BCF3 and was demonstrated to be a reversible inhibitor of GABA-AT. The kinetic studies and computational calculations provide evidence to support that the conjugated alkene of SS-1-148 in its cyclopentene ring allows for retaining high hOAT inactivation efficiency. Two crystallographic approaches were performed to capture a temporary gem-diamine intermediate covalently bond with Lys292 in the soaked crystal and a stable non-covalent final product in the cocrystal complex. The critical salt bridge of Arg413-Glu235 in hOAT is found to be broken in both crystal complexes. Moreover, native/intact MS experiments further support a non-covalent pathway as the primary inactivation mechanism of SS-1-148 and a covalent modification observed as a minor form that can be a gem-diamine structure in equilibrium with the non-covalent form. In the end, stopped-flow experiments suggest that the first external aldimine of SS-1-148 undergoes an unusual E2 fluoride ion elimination instead of the typical E1CB elimination reaction, forming the second external aldimine as the single transient state. Comprehensive mechanistic studies demonstrate that SS-1-148 mainly inactivates hOAT non-covalently through a water-mediated mechanism. In addition, the preliminary assessment suggests that SS-1-148 exhibits favorable DMPK properties (data not shown), and it is being investigated in the PDX mouse models of HCC.

### ABBREVIATIONS

AFPA, (S)-4-amino-5-fluoropentanoic acid; Boc₂O, di-tert-butyl dicarbonate; Deoxo-Fluor, bis(2-methoxyethyl)aminosulfur trifluoride; DMPK, drug metabolism and pharmacokinetics; DIPEA, N,N-diisopropylethylamine; DBDMH. 1,3-dibromo-5,5-dimethylhydantoin; DMF, dimethylformamide; DMSO, dimethyl sulfoxide; DCM, dichloromethane; IND, investigational new drug application; PO, Per os administration; THE, tetrahydrofuran; TEA, triethylamine; TBAI, tetra-n-butylammonium iodide.

### REFERENCES

1. Lee, H ; Juncosa, J. I.; Silverman, R. B., Ornithine aminotransferase versus GABA aminotransferase: implications for the design of new anticancer drugs. Med Res Rev 2015, 35 (2), 286-305.
2. Tanner, J. J.; Fendt, S. M.; Becker, D. F., The Proline Cycle As a Potential Cancer Therapy Target. Biochemistry 2018, 57 (25), 3433-3444.
3. Tang, L.; Zeng, J.; Geng, P.; Fang, C.; Wang, Y.; Sun, M.; Wang, C.; Wang, J.; Yin, P.; Hu, C.; Guo, L.; Yu, J.; Gao, P.; Li, E.; Zhuang, Z.; Xu, G.; Liu, Y., Global Metabolic Profiling Identifies a Pivotal Role of Proline and Hydroxyproline Metabolism in Supporting Hypoxic Response in Hepatocellular Carcinoma. Clin Cancer Res 2018, 24 (2), 474-485.
4. Cadoret, A.; Ovejero, C.; Terris, B.; Souil, E.; Levy, L.; Lamers, W. H.; Kitajewski, J.; Kahn, A.; Perret, C. , New targets of beta-catenin signaling in the liver are involved in the glutamine metabolism. Oncogene 2002, 21 (54), 8293-301.
5. Zigmond, E.; Ben Ya'acov, A.; Lee, H.; Lichtenstein, Y.; Shalev, Z.; Smith, Y.; Zolotarov, L.; Ziv, E.; Kalman, R.; Le, H. V.; Lu, H.; Silverman, R. B.; Ilan, Y., Suppression of Hepatocellular Carcinoma by Inhibition of Overexpressed Ornithine Aminotransferase. ACS Med Chem Lett 2015, 6 (8), 840-4.
6. Eliot, A. C.; Kirsch, J. F., Pyridoxal phosphate enzymes: mechanistic, structural, and evolutionary considerations. Annu Rev Biochem 2004, 73, 383-415.
7. Silverman, R. B., Design and Mechanism of GABA Aminotransferase Inactivators. Treatments for Epilepsies and Addictions. Chem Rev 2018, 118 (7), 4037-4070.
8. Pan, Y.; Gerasimov, M. R.; Kvist, T.; Wellendorph, P.; Madsen, K. K.; Pera, E.; Lee, H., Schousboe, A.; Chebib, M.; Brauner-Osborne, H.; Craft, C. M.; Brodie, J. D.; Schiffer, W. K.; Dewey, S. L.; Miller, S. R.; Silverman, R. B., (1S, 3S)-3-amino-4-difluoromethylenyl-1-cyclopentanoic acid (CPP-115), a potent gamma-aminobutyric acid aminotransferase inactivator for the treatment of cocaine addiction. J Med Chem 2012, 55 (1), 357-66.

9 Moschitto, M. J.; Doubleday, P. F.; Catlin, D. S.; Kelleher, N. L.; Liu, D.; Silverman, R. B., Mechanism of Inactivation of Ornithine Aminotransferase by (1S,3S)-3-Amino-4-(hexafluoropropan-2-ylidenyl)cyclopentane-1-carboxylic Acid. J Am Chem Soc 2019, 141 (27), 10711-10721.

10. Butrin, A.; Beaupre, B. A.; Kadamandla, N.; Zhao, P.; Shen, S.; Silverman, R. B.; Moran, G. R.; Liu, D., Structural and Kinetic Analyses Reveal the Dual Inhibition Modes of Ornithine Aminotransferase by (1S,3S)-3-Amino-4-(hexafluoropropan-2-ylidenyl)-cyclopentane-1-carboxylic Acid (BCF3). ACS Chemical Biology 2020.
11. Zhu. W.; Doubleday, P. F.; Catlin, D. S.; Weerawarna, P. M.; Butrin, A.; Shen, S.; Wawrzak, Z.; Kelleher, N. L.; Liu, D.; Silverman, R. B., A Remarkable Difference That One Fluorine Atom Confers on the Mechanisms of Inactivation of Human Ornithine Aminotransferase by Two Cyclohexene Analogues of gamma-Aminobutyric Acid. J Am Chem Soc 2020, 742 (10), 4892-4903.
12. Daune, G.; Gerhart, F.; Seiler, N., 5-Fluoromethylornithine, an irreversible and specific inhibitor of L-ornithine:2-oxo-acid aminotransferase. Biochem J 1988, 253 (2), 481-8.
13. Bolkenius, F. N.; Knodgen, B.; Seiler, N., DL-canaline and 5-fluoromethylornithine. Comparison of two inactivators of ornithine aminotransferase. Biochem J 1990, 268 (2), 409-14.
14. Storici, P.; Capitani, G.; Muller, R.; Schirmer, T.; Jansonius, J. N., Crystal structure of human ornithine aminotransferase complexed with the highly specific and potent inhibitor 5-fluoromethylornithine. J Mol Biol 1999, 285 (1). 297-309.
15. Qiu, J.; Silverman, R. B., A new class of conformationally rigid analogues of 4-amino-5-halopentanoic acids, potent inactivators of gamma-aminobutyric acid aminotransferase. J Med Chem 2000, 43 (4), 706-20.
16. Juncosa, J. I.; Takaya, K.; Le, H. V.; Moschitto, M. J.; Weerawarna, P. M.; Mascarenhas, R.; Liu, D.; Dewey, S. L.; Silverman, R. B., Design and Mechanism of (S)-3-Amino-4-(difluoromethylenyl)cyclopent-1-ene-1-carboxylic Acid, a Highly Potent gamma-Aminobutyric Acid Aminotransferase Inactivator for the Treatment of Addiction. J Am Chem Soc 2018, 140 (6), 2151-2164.
17. Shen, S.; Doubleday, P. F.; Weerawarna, P. M.; Zhu, W.; Kelleher, N. L.; Silverman, R. B., Mechanism-Based Design of 3-Amino-4-Halocyclopentenecarboxylic Acids as Inactivators of GABA Aminotransferase. ACS Med Chem Leit 2020, 11 (10), 1949-1955.
18. Singh. R.; Vince, R., 2-Azabicyclo[2.2.1]hept-5-en-3-one: chemical profile of a versatile synthetic building block and its impact on the development of therapeutics. Chem Rev 2012, 112 (8), 4642-86.
19. Wang, B. L.; Gao, H. T.; Li, W. D., Total synthesis of (+)-iresin. J Org Chem 2015, 80 (10), 5296-301.
20. Gross, L. J.; Stark, C. B. W., Regioselective dehydration of alpha-hydroxymethyl tetrahydrofurans using Burgess' reagent under microwave irradiation. Org Biomol Chem 2017, 15 (20), 4282-4285.
21. Lee, H.; Doud, E. H.; Wu, R.; Sanishvili, R.; Juncosa, J. I.; Liu, D.; Kelleher, N. L.; Silverman, R. B., Mechanism of inactivation of gamma-aminobutyric acid aminotransferase by (1S,3S)-3-amino-4-difluoromethylene-1-cyclopentanoic acid (CPP-115). J Am Chem Soc 2015, 137 (7), 2628-40.
22 Shen, S.; Doubleday, P. F.; Weerawarna, P. M.; Zhu, W.; Kelleher, N. L.; Silverman, R. B., Mechanism-Based Design of 3-Amino-4-Halocyclopentenecarboxylic Acids as Inactivators of GABA Aminotransferase. ACS Medicinal Chemistry Letters 2020.
23. Mascarenhas, R.; Le, H. V.; Clevenger, K. D.; Lehrer, H. J.; Ringe, D.; Kelleher, N. L.; Silverman, R. B.; Liu, D., Selective Targeting by a Mechanism-Based Inactivator against Pyridoxal 5′-Phosphate-Dependent Enzymes: Mechanisms of Inactivation and Alternative Turnover. Biochemistry 2017, 56 (37), 4951-4961.
24. Di Salvo, M. L.; Scarsdale, J. N.; Kazanina, G.; Contestabile, R.; Schirch, V.; Wright, H. T., Structure-based mechanism for early PLP-mediated steps of rabbit cytosolic serine hydroxymethyltransferase reaction. Biomed Res Int 2013, 2013, 458571.
25. Soniya, K.; Awasthi, S.; Nair, N. N.; Chandra, A., Transimination Reaction at the Active Site of Aspartate Aminotransferase: A Proton Hopping Mechanism through Pyridoxal 5′-Phosphate. ACS Catalysis 2019, 9 (7), 6276-6283.
26. Shah, S. A.; Shen, B. W; Brunger, A. T., Human ornithine aminotransferase complexed with L-canaline and gabaculine: structural basis for substrate recognition. Structure 1997, 5 (8), 1067-75.
27. Leney, A. C.; Heck, A. J., Native Mass Spectrometry: What is in the Name? J Am Soc Mass Spectrom 2017, 28 (1), 5-13.
28. Stewart, J. J., MOPAC: a semiempirical molecular orbital program. J Comput Aided Mol Des 1990, 4 (1), 1-105.
29. Karsten, W. E.; Ohshiro, T.; Izumi, Y.; Cook, P. F., Reaction of serine-glyoxylate aminotransferase with the alternative substrate ketomalonate indicates rate-limiting protonation of a quinonoid intermediate. Biochemistry 2005, 44 (48), 15930-6.
30. Thibodeaux, C. J.; Liu, H. W., Mechanistic studies of 1-aminocyclopropane-1-carboxylate deaminase: characterization of an unusual pyridoxal 5′-phosphate-dependent reaction. Biochemistry 2011, 50 (11), 1950-62.
31. Clift, M. D.; Ji, H.; Deniau, G. P.; O'Hagan, D.; Silverman, R. B., Enantiomers of 4-amino-3-fluorobutanoic acid as substrates for gamma-aminobutyric acid aminotransferase. Conformational probes for GABA binding. Biochemistry 2007, 46 (48), 13819-28.
32. Gokcan, H.; Konuklar, F. A., Theoretical study on HF elimination and aromatization mechanisms: a case of pyridoxal 5′ phosphate-dependent enzyme. J Org Chem 2012, 77 (13), 5533-43.
33. Alunni, S.; De Angelis, F.; Ottavi, L.; Papavasileiou, M.; Tarantelli, F., Evidence of a borderline region between E1cb and E2 elimination reaction mechanisms: a combined experimental and theoretical study of systems activated by the pyridine ring. J Am Chem Soc 2005, 127 (43), 15151-60.
34. Ghalami-Choobar, B.; Dezhampanah, H.; Nikparsa, P.; Ghiami-Shomami, A., Theoretical calculation of the pKa values of some drugs in aqueous solution. International Journal of Quantum Chemistry 2012, 112 (10), 2275-2280.

### Example 2—DMPK Results for Example 1

TABLE 2
Drug-like properties of SS-1-148^{a}

| Physiochemical properties | | | | Plasma PK profiles | | | |
|---|---|---|---|---|---|---|---|
| MW | 163.12 | tPSA | 63.3 | IV (5 mg/kg) | Cₘₐₓ | ng/ml | 4677 |
| acidic pKₐ | 3.66 | clogP | −2.28 | | AUCₗₐₛₜ | h*ng/ml | 1147 |
| basic pKₐ | 7.90 | clogD (7.4) | −2.59 | | AUC_{inf} | h*ng/ml | 1158 |

| aqueous solubility | >50 mM (HCl salt) | | | | T_{1/2} | H | 0.43 |
|---|---|---|---|---|---|---|---|
| In vitro ADMET | | | | | Cl | mL/min/kg | 72 |
| Off-target effects | Asp-AT | >20 | | | Vₛₛ | l/kg | 1.01 |
| (Ki, mM) | Ala-AT | >20 | PO (30 mg/kg) | | Tₘₐₓ | h | 0.25 |
| Liver microsomal stability | Mouse | N/C | | | Cₘₐₓ | ng/ml | 8076 |
| (t_{1/2}) | Human | N/C | | | AUCₗₐₛₜ | h*ng/ml | 8445 |
| Plasma protein binding | Mouse | 0% | | | AUC_{inf} | h*ng/ml | 8526 |
| | Human | 0% | | | F % | — | ~100 |
| hERG test (IC₅₀, μM) | | >30 | Brain/plasma PK studies | | | | |
| CYP inhibition | 1A2 | >100 | Route | Time | Brain con. | Plasma con. | B/P ratio |
| (IC₅₀, μM) | | | | (h) | (ng/g) | (ng/mL) | |
| | 2D6 | >100 | IV (5 mg/kg) | 0.25 | 353 ± 11 | 1766 ± 166 | 0.20 |
| | 2C9 | >100 | | 2 | n/a | n/a | n/a |
| | 2C19 | >100 | PO (30 mg/kg) | 0.25 | 8075 ± 2252 | 1311 ± 346 | 0.14 |
| | 3A4 | >100 | | 2 | 812 ± 404 | 215 ± 68 | 0.26 |

^{a}In vitro and in vivo ADMET profiles were generated by Sai Life Sciences Limited (India)

### Example 3—Supplementary Material for Example 1

### Supplementary Tables

TABLE 3
Statistics of the crystal structures of hOAT inactivated by SS-1-148

| Complex | Cocrystal | Soaking |
|---|---|---|
| PDB code | 7LK0 | 7LK1 |
| Space group | P 3₁ 1 2 | P 3₂ 2 1 |

Unit Cell dimension

| | | |
|---|---|---|
| α, β, γ (deg) | 90, 90, 120 | 90, 90, 120 |
| a, b, c (Å) | 192.7, 192.7, 56.8 | 115.6, 115.6, 186.6 |
| Processed Resolution (Å) | 1.96 | 1.79 |
| R_{merge} ^{a} (%) | 16.9 (81.7) | 8.4 (248.7) |
| Rₚᵢₘ ^{c} (%) | 8.3 (60.4) | 2.8 (83.3) |
| I/σ (I) | 5.6 (1.1) | 13.7 (0.9) |
| CC½ ^{d} (%) | 99.7 (78.3) | 99.9 (34.4) |
| Completeness (%) | 91.6 (72.1) | 100.0 (100.0) |
| Multiplicity | 8.7 (4.1) | 9.6 (9.8) |
| No. Reflections | 685516 | 1311708 |
| No. Unique Reflections | 79187 | 136181 |

Refinement

| | | |
|---|---|---|
| R_{work} ^{e}/R_{free} ^{f} (%) | 24.54/26.60 | 21.60/24.80 |

No. of Atoms

| | | |
|---|---|---|
| protein | 9462 | 9444 |
| ligand | 75 | 170 |
| water | 582 | 766 |

Average B factors (Å²)

| | | |
|---|---|---|
| protein | 37.17 | 44.34 |

RMSD^{g}

| | | |
|---|---|---|
| bond lengths (Å) | 0.004 | 0.005 |
| bond angles (deg) | 0.69 | 0.84 |

Ramachandran plot (%)

| | | |
|---|---|---|
| favored | 94.75 | 94.90 |
| allowed | 5.16 | 4.93 |
| outliers | 0.08 | 0.17 |

^{a} R_{merge} = Σ|I_{obs} − I_{avg}|ΣI_{avg},
^{b}The values for the highest-resolution bin are in parentheses,
^{c} Precision-indicating merging R,
^{d} Pearson correlation coefficient of two “half” data sets,
^{e} R_{work} = Σ|F_{obs} − F_{calc}|/ΣF_{obs},
^{f} Five percent of the reflection data were selected at random as a test set, and only these data were used to calculate R_{free},
^{g}Root-mean square deviation.

### Synthesis of 4-6

### General Procedure

Commercially available reagents and solvents were used without further purification. All reactions were monitored by thin-layer chromatography (TLC) using 0.25 mm SiliCycle extra hard 250 μM TLC plates (60 F₂₅₄), and spots were visualized under UV (254 nm) and ceric ammonium molybdate or ninhydrin stain. Flash chromatography was performed on a Combi-Flash Rf system (Teledyne ISCO) with silica columns and reversed-phase C-18 columns. Analytical HPLC was used to determine the purity of all of the final products using an Agilent 1260 series instrument with the following conditions: column, Phenomenex Kintex C-18 column (50×2.1 mm, 2.6 μm): mobile phase, 5-100% acetonitrile/water containing 0.05% TFA at a flow rate of 0.9 mL/min for 6 min; UV detection at 254 nm. The purity of all tested compounds for in vitro biological studies was >95% by HPLC analysis. ¹H, ¹³C, and 2D NMR spectra were obtained using Bruker AVANCE III 500 MHz system and Bruker NEO console w/QCI-F cryoprobe 600 MHz system. Chemical shifts were reported relative to CDCl₃ (δ=7.26 for ¹H NMR and δ=77.16 for ¹³C NMR spectra), CD₃OD (δ=3.31 for ¹H NMR and δ=49.15 for ¹³C NMR spectra), and DMSO-d₆ (δ=2.50 for ¹H NMR and δ=39.52 for ¹³C NMR spectra). The following abbreviations for multiplicities were used: s=singlet, d=doublet, t=triplet, q=quartet, m=multiplet, dd=doublet of doublets, dt=doublet of triplets, dq=doublet of quartets, ddt=doublet of doublet of triplets, ddd=doublet of doublet of doublets, dddd=doublet of doublet of doublet of doublets, dddt=doublet of doublet of doublet of triplets, and br s=broad singlet. Low-resolution mass spectra (LRMS) were obtained using a Thermo TSQ Quantum system in the positive ion mode using atmospheric pressure chemical ionization (APCI) with the Agilent Infinity 1260 HPLC system abovementioned. High resolution mass spectra (HRMS) were obtained on an Agilent 6210 LC-TOF spectrometer in the positive ion mode using electrospray ionization (ESI) with an Agilent G1312A HPLC pump and an Agilent G1367B autoinjector at the Integrated Molecular Structure Education and Research Center (IMSERC), Northwestern University.

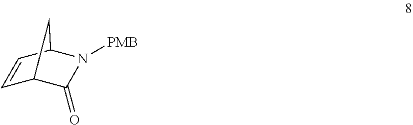

(1R,4S)-2-(4-Methoxybenzyl)-2-azabicyclo[2.2.1]hept-5-en-3-one (8).² (i) p-Anisyl alcohol (10 mL) and conc. HCl (15 mL) were dissolved in a 100 mL round-bottom three necked flask at room temperature. The resulting mixture was stirred at room temperature for an additional 1 h. After the completion of the reaction, the solution was poured into ice and extracted with EtOAc (20 mL×3). The combined organic layers were separated, washed with sat. Na₂CO₃ solution and brine, dried over Na₂SO₄, and concentrated under vacuum. Fresh 4-methoxybenzyl chloride was obtained as a colorless oil (9.2 g, 52%) and was used directly in the next step without further purification. (ii) To a stirred solution of (1R)-(−)-2-azabicyclo[2.2.1]hept-5-en-3-one (1, CAS #79200-56-9, 5.8 g. 53 mmol) in dry THF (300 mL) was added NaH (60%, 3.18 g, 80 mmol) suspended in DMF (30 mL) in an ice bath. The resulting mixture was stirred at the same temperature for 30 min, followed by the addition of 4-methoxybenzyl chloride (9.2 g, 63.6 mmol) and TBAI (1.96 g, 5.3 mmol) at 0° C. The resulting mixture was slowly warmed to room temperature and stirred for an additional 3 h. After the completion of the reaction, the solution was quenched with water (200 mL) and extracted with EtOAc (100 mL×3). The combined organic layers were separated, washed with sat. Na₂CO₃ solution and brine, dried over Na₂SO₄, and concentrated under vacuum. The crude product was purified via Combi-Flash chromatography (EtOAc/hexane: 0-50%) to yield 8 (7.8 g, 65%) as a colorless oil. ¹H NMR (500 MHz, CD₃OD) δ 7.12 (d, J=8.5 Hz, 2H), 6.87 (d, J=8.6 Hz, 2H), 6.62-6.50 (m, 2H), 4.24 (d, J=14.6 Hz, 1H), 4.16 (q, J=1.9 Hz, 1H), 4.10 (d, J=14.6 Hz, 1H), 3.77 (s, 3H), 3.32-3.30 (m, 1H), 2.25 (dt, J=7.7, 1.8 Hz, 1H), 2.07 (dt, J=7.7, 1.6 Hz, 1H). ¹³C NMR (126 MHz, CD₃OD) δ 182.5, 160.8, 141.6, 137.6, 130.9 (2C), 129.2, 115.0 (2C), 64.6, 60.0, 55.9, 55.1, 47.8. LRMS (APCI) calcd for C₁₄H₁₆NO₂ [M+H]⁺: 230.12; found, 230.21; T_{R}=2.28 min.

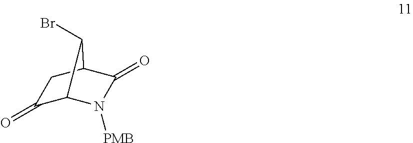

(1R,4R,7R)-7-Bromo-2-(4-methoxybenzyl)-2-azabicyclo[2.2.1]heptane-3,6-dione (11). To a stirred solution of oxalyl chloride (3.69 mL, 42.9 mmol) in DCM (200 mL) was slowly added DMSO (5.0 mL, 70.6 mmol) at −78° C. under argon atmosphere. After stirring at −78° C. for 10 min, 10¹ (10.0 g, 30.7 mmol) dissolved in DCM (200 mL) was added to the resulting mixture at the same temperature followed by stirring at −78° C. for additional 10 min TEA (30 mL, 215 mmol) was then added dropwise. Upon completion of the addition, the reaction was stirred at −78° C. for 10 min, warmed to room temperature, and quenched with 1 N NH₄Cl. The organic layer was separated, washed with brine, dried over Na₂SO₄, and concentrated under vacuum. The crude product was washed with EtOH to yield 11 (7.8 g, 73%) as a brown solid used in the next step without further purification. ¹H NMR (500 MHz, CDCl₃) δ 7.22-6.99 (m, 2H), 6.92-6.75 (m, 2H), 4.72 (d, J=14.7 Hz, 1H), 4.35 (d, J=2.3 Hz, 1H), 3.94 (d, J=14.7 Hz, 1H), 3.80 (s, 3H), 3.66 (d, J=2.0 Hz, 1H), 3.16 (dt, J=4.1, 2.0 Hz, 1H), 2.71 (dd, J=178, 4.2 Hz, 1H), 2.22 (dd, J=17.9, 2.5 Hz, 1H). ¹³C NMR (126 MHz, CDCl) δ 202.9, 172.6, 159.6, 129.8 (2C), 127.0, 114.4 (2C), 68.4, 55.3, 49.0, 48.2, 45.3, 32.1. LRMS (APCI) calcd for C₁₄H₁₅BrNO₃ [M+H]⁺: 324.02; found, 324.33; T_{R}=2.40 min.

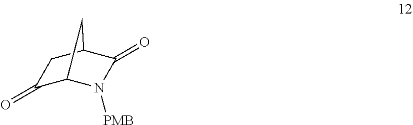

(1S,4R)-2-(4-Methoxybenzyl)-2-azabicyclo[2.2.1]heptane-3,6-dione (12). To a stirred solution of 11 (6.0 g, 18.5 mmol) in benzene (100 mL) were added Bn₃SnH (7 mL, 26.0 mmol) and AIBN (151 mg, 0.925 mmol) at room temperature. Then the resulting mixture was heated to reflux overnight. After completion of the reaction, the solution was quenched with water and extracted with EtOAc (100 mL×3) The combined organic layers were separated, washed with brine, dried over Na₂SO₄, and concentrated under vacuum. The residue was purified via Combi-Flash chromatography (EtOAc/hexane: 0-50%) to obtain 12 (3.1 g, 68%) as an off-white solid. ¹H NMR (500 MHz, CDCl₃) δ 7.22-7.13 (m, 2H), 6.89-6.80 (m, 2H), 4.70 (d, J=14.8 Hz, 1H), 3.88 (d, J=14.8 Hz, 1H), 3.80 (d, J=1.1 Hz, 3H), 3.56 (q, J=1.7 Hz, 1H), 3.03 (qd, J=2.3, 1.4 Hz, 1H), 2.30-2.22 (m, 1H), 2.18 (td, J=4.0, 3.4, 2.1 Hz, 2H), 1.86 (dt, J=10.7, 1.5 Hz, 1H). ¹³C NMR (126 MHz, CDCl₃) δ 206.2, 176.6, 159.4, 129.7 (2C), 127.9, 114.2 (2C), 64.6, 55.3, 45.1, 42.5, 39.1, 35.2. LRMS (APCI) calcd for C₁₄H₁₆NO₃ [M+H]⁺: 246.11; found, 246.10; T_{R}=1.81 min.

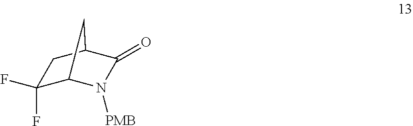

(1S,4R)-6,6-Difluoro-2-(4-methoxybenzyl)-2-azabicyclo[2.2.1]heptan-3-one (13). 12 (245 mg, 1.0 mmol) was dissolved in THF (3 mL) in a microwave tube, then Deoxo-Fluor (1.11 mL, 3.0 mmol, 2.7 M in toluene) was added at room temperature. The resulting mixture was heated at 120° C. for 40 min in a microwave reactor. After completion of the reaction, the solution was quenched with sat. NaHCO₃ solution and extracted with EtOAc (10 mL×3). The combined organic layers were separated, washed with brine, dried over Na₂SO₄, and concentrated under vacuum. The residue was purified via Combi-Flash chromatography (EtO Ac/hexane: 0-100%) to obtain 13 (190 mg, 71%) as a colorless oil. ¹H NMR (500 MHz, CDCl₃) δ 7.26-7.12 (m, 2H), 6.99-6.73 (m, 2H), 4.99 (d, J=14.9 Hz, 1H), 3.81 (s, 3H), 3.78 (s, 1H), 3.61 (d, J=2.0 Hz, 1H), 2.85 (dq, J=3.9, 1.8 Hz, 1H), 2.32-2.10 (m, 2H), 2.01 (dtd, J=10.4, 3.6, 1.8 Hz, 1H), 1.85 (ddd, J=10.6, 3.8, 1.9 Hz, 1H). ¹³C NMR (126 MHz, CDCl₃) δ 175.9, 159.2, 129.5 (2C), 130.05 (t, J=260.4 Hz), 128.2, 114.2 (2C), 61.47 (dd, J=32.7, 21.0 Hz), 45.35 (d, J=2.7 Hz), 43.69 (t, J=4.0 Hz), 38.86 (d, J=3.9 Hz), 36.7 (t, J=23.9 Hz). LRMS (APCI) calcd for C₁₄H₁₆F₂NO₂ [M+H]⁺: 268.11; found, 268.07; T_{R}=2.36 min.

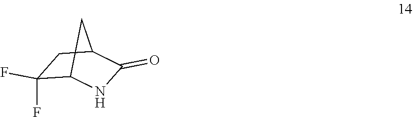

(1S,4R)-6,6-Difluoro-2-azabicyclo[2.2.1]heptan-3-one (14). To a stirred solution of 13 (190 mg, 0.71 mmol) in CH₃CN (8 mL) was added an aqueous solution of ceric ammonium nitrate (1.16 g in 3 mL water, 2.10 mmol) at room temperature. The resulting mixture was stirred for 2 h until the starting material completely disappeared. The residue was extracted with EtOAc (10 mL×3). The combined organic layers were separated, washed with sat. Na₂CO₃ solution and brine, dried over Na₂SO₄, and concentrated under vacuum. The crude product was purified via Combi-Flash chromatography (EtOAc/hexane: 0-100%) to obtain 14 (60 mg, 57%) as a white powder. ¹H NMR (500 MHz, CDCl₃) δ 5.69 (br s, 1H), 3.82 (t, J=1.9 Hz, 1H), 2.77 (dd, J=4.5, 2.3 Hz, 1H), 2.35-2.21 (m, 1H), 2.23-2.16 (m, 1H), 2.17-2.03 (m, 1H), 1.98 (ddq, J=10.5, 3.6, 1.7 Hz, 1H). ¹³C NMR (126 MHz, CDCl₃) δ 179.0, 128.8 (t, J=258.6 Hz), 59.4 (dd, J=33.2, 22.6 Hz), 43.6 (t, J=4.0 Hz), 39.1 (d, J=3.1 Hz), 36.97 (dd, J=25.1, 23.4 Hz). LRMS (APCI) calcd for C₆H₈F₂NO [M+H]⁺: 148.06; found, 148.02; T_{R}=0.33 min.

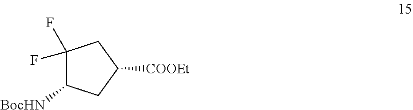 [SMILES: CCOC(=O)[C@@H]1C[C@H](NC(=O)OC(C)(C)C)C(F)(F)C1]

(1R,4S)-Ethyl 4-((tert-butoxycarbonyl)amino)-3,3-difluorocyclopentanecarboxylate (15). (i) 14 (150 mg, 1.02 mmol) was dissolved in HCl ethanol solution (2 mL, 1.2 M) at room temperature. The resulting mixture was heated at 85° C. overnight in a pressure tube. After the completion of the reaction, the excess solvent was removed under vacuum. The crude product was obtained as a light yellow solid and used directly to the next step without further purification. (ii) To a stirred solution of the intermediate in MeOH (5 mL) were added Boc₂O (327 mg, 1.50 mmol) and TEA (0.21 mL, 1.50 mmol) at room temperature. The resulting mixture was stirred at room temperature for 2 h. After the completion of the reaction, the excess solvent was removed under vacuum, and the residue was quenched with water and extracted with EtOAc (25 mL×3). The combined organic layers were separated, washed with brine, dried over Na₂SO₄, and concentrated under vacuum. The crude product was purified via Combi-Flash chromatography (EtOAc/hexane: 0-100%) to obtain 15 as a white solid (240 mg, 82% over two steps). ¹H NMR (500 MHz, CDCl₃) δ 5.14-4.64 (m, 1H), 4.27-4.19 (m, 1H), 4.16 (q, J=7.2 Hz, 2H), 3.01-2.85 (m, 1H), 2.56-2.32 (m, 3H), 1.77 (dtd, J=12.1, 10.0, 1.8 Hz, 1H), 1.45 (s, 9H), 1.26 (t, J=7.1 Hz, 3H). ¹³C NMR (126 MHz, CDCl₃) δ 173.5, 155.1, 127.2 (dd, J=255.6, 250.9 Hz), 80.2, 61.7, 55.2 (dd, J=26.0, 20.6 Hz), 36.3, 35.8 (t, J=25.8 Hz), 32.9 (d, J=4.5 Hz), 28.3 (3C), 14.1. LRMS (APCI) calcd for C₈H₁₄F₂NO₂ [M-Boc+2H]⁺: 194.10; found, 193.99; T_{R}=2.67 min.

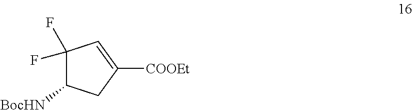 [SMILES: CCOC(=O)C1=CC(F)(F)[C@@H](NC(=O)OC(C)(C)C)C1]

(S)-Ethyl 4-((tert-Butoxycarbonyl)amino)-3,3-difluorocyclopent-1-enecarboxylate (16). To a stirred solution of 15 (100 mg, 0.34 mmol) in THF (8 mL) was added KHMDS (2.0 mL, 0.5 M in toluene) at −78° C. The resulting mixture was allowed to stir at −78° C. for 2 h followed by the addition of phenylselenyl chloride (78 mg, 0.41 mmol) in a THF (2 mL) solution at the temperature. The reaction was warmed to room temperature slowly and stirred for an additional 6 h. The reaction was quenched with saturated aqueous NH₄Cl and extracted with EtOAc (10 mL×3). The combined organic layers were separated, washed with brine, dried over Na₂SO₄, and concentrated under vacuum. The crude product was purified via Combi-Flash chromatography (EtOAc/hexane: 0-100%) to obtain 16 (20 mg, 20%) as a white solid. ¹H NMR (500 MHz, CDCl₃) δ 6.60 (s, 1H), 4.91 (d, J=9.0 Hz, 1H), 4.57-4.53 (m, 1H), 4.26 (q, J=7.1 Hz, 2H), 3.17 (dt, J=16.1, 7.4 Hz, 1H), 2.42 (ddt, J=14.8, 6.1, 3.0 Hz, 1H), 1.47 (s, 9H), 1.32 (t, J=7.1 Hz, 3H). ¹³C NMR (126 MHz, CDCl₃) δ 163.1, 155.1, 143.1 (t, J=10.1 Hz), 132.44 (dd, J=30.7, 24.6 Hz), 127.2 (t, J=251.0 Hz), 80.4, 61.5, 54.5 (t, J=20.9 Hz), 35.8, 28.3 (3C), 14.1. LRMS (APCI) calcd for C₈H₁₂F₂NO₂ [M-Boc+2H]⁺: 192.08; found, 191.96; T_{R}=2.71 min.

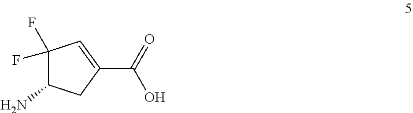 [SMILES: N[C@H]1CC(C(=O)O)=CC1(F)F]

(S)-4-Amino-3,3-difluorocyclopent-1-enecarboxylic acid (5). To a stirred solution of 16 (20 mg, 0.07 mmol) in AcOH (1 mL) was added 4N HCl (1 mL) at room temperature. The resulting mixture was heated at 70° C. overnight. After the completion of the reaction, the excess solvent was removed under vacuum. The crude product was purified via Combi-Flash chromatography (C18 Reverse Column, CH₃CN/H₂O: 0-5%) to obtain 5 as a white powder HCl salt (10 mg, 71%). ¹H NMR (600 MHz, CD₃OD) δ 6.65 (s, 1H), 4.22 (dq, J=13.2, 7.1, 6.7 Hz, 1H), 3.28-3.21 (m, 1H), 2.80-2.64 (m, 1H). ¹³C NMR (126 MHz, CD₃OD) δ 165.23, 145.84 (dd, J=11.3, 9.5 Hz), 131.65 (dd, J=29.4, 25.5 Hz), 129.21 (t, J=248.6 Hz), 54.50 (dd, J=31.3, 20.0 Hz), 34.64 (d, J=2.2 Hz). HRMS (ESI) calcd for C₆H₈FNO₂ [M+H]⁺: 164.0518; found, 164.0516.

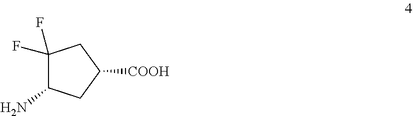 [SMILES: N[C@H]1C[C@@H](C(=O)O)CC1(F)F]

(1R,4S)-4-Amino-3,3-difluorocyclopentanecarboxylic acid hydrochloric acid salt (4). 14 (60 mg, 0.41 mmol) was dissolved in 4N HCl (4 mL) at room temperature. The resulting mixture was heated at 70° C. for 1 h. After the completion of the reaction, the excess solvent was removed under vacuum. The crude product was purified via Combi-Flash chromatography (C18 Reverse Column, CH₃CN/H₂O: 0-5%) to obtain 4 as a white powder HCl salt (60 mg, 89%). ¹H NMR (500 MHz, CD₃OD) δ 3.93 (qd, J=11.4, 7.8 Hz, 1H), 3.16 (tt, J=10.0, 8.2 Hz, 1H), 2.69-2.47 (m, 3H), 2.04 (dtd, J=12.3, 10.7, 1.4 Hz, 1H). ¹³C NMR (126 MHz, CD₃OD) δ 175.5, 128.3 (dd, J=255.0, 252.6 Hz), 55.8 (dd, J=29.6, 20.7 Hz), 37.9 (t, J=4.5 Hz), 37.2 (t, J=24.8 Hz), 31.9 (d, J=4.8 Hz). HRMS (ESI) calcd for C₆H₁₀FNO₂ [M+H]⁺: 166.0674; found, 166.0671.

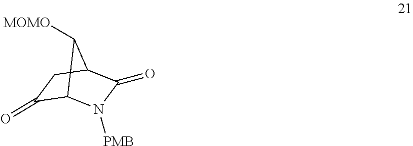

(1S,4S,7R)-2-(4-Methoxybenzyl)-7-(methoxymethoxy)-2-azabicyclo[2.2.1]heptane-3,6-dione (21) was prepared from 20² (12.0 g, 39 mmol) following the procedure for the synthesis of 12 to afford as a colorless oil (10.8 g, 91%). ¹H NMR (500 MHz, CDCl;) δ 7.20 -7.04 (m, 2H), 6.97-6.77 (m, 2H), 4.64 (d, J=6.9 Hz, 1H), 4.62 (d, J=14.8 Hz, 1H), 4.59 (d, J=6.9 Hz, 1H), 4.17 (q, J=2.2 Hz, 1H), 4.02 (d, J=14.8 Hz, 1H), 3 80 (s, 3H), 3.59 (t, J=1.9 Hz, 1H), 3.30 (d, J=1.7 Hz, 3H), 3.11-3.05 (m, 1H), 2.51 (dd, J=17.5, 4.3 Hz, 1H), 2.14 (dd, J=17.4, 1.9 Hz, 1H). ¹³C NMR (126 MHz, CDCl₃) δ 206.0, 173.4, 159.4, 129.7 (2C), 127.7, 114.3 (2C), 96.2, 80.5, 66.6, 56.3, 55.3, 46.9, 45.0, 31.5. LRMS (APCI) calcd for C₁₆H₂₀NO₅ [M+H]⁺: 306.13; found, 306.39; T_{R}=2.36 min.

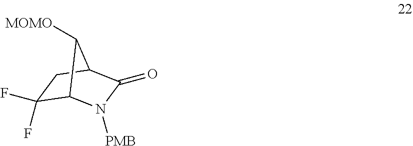

(1S,4S,7R)-6,6-Difluoro-2-(4-methoxybenzyl)-7-(methoxymethoxy)-2-azabicyclo[2.2.1]-heptan-3-one (18) was synthesized from 17 (670 mg, 2.20 mmol) following the procedure for the synthesis of 13 to afford as a brown oil (490 mg, 72%). ¹H NMR (500 MHz, CDCl₃) δ 7.15 (d, J=8.6 Hz, 2H), 6.87 (d, J=8.6 Hz, 2H), 5.02 (d, J=14.9 Hz, 1H), 4.59 (s, 3H), 4.03 (dt, J=4.7, 2.0 Hz, 1H), 3.80 (s, 3H), 3.79 (d, J=14.9 Hz, 1H), 3.58 (q, J=1.8 Hz, 1H), 3.48-3.34 (m, 2H), 3.33 (s, 3H), 2.96-2.84 (m, 1H), 2.57 (dddd, J=18.0, 13.8, 9.4, 4.3 Hz, 1H), 2.16 (dddd, J=15.9, 13.7, 6.2, 1.7 Hz, 1H). ¹³C NMR (126 MHz, CDCl₃) δ 172.5, 159.3, 129.72 (dd, J=274, 254 Hz), 129.6 (2C), 127.8, 114.3 (2C), 95.9, 81.2 (d, J=4.0 Hz), 62.4 (dd, J=29.9, 20.3 Hz), 56.2 (d, J=1.7 Hz), 55.3, 48.5 (t, J=3.8 Hz), 45.3 (d, J=2.6 Hz), 34.8 (t, J=24.7 Hz). LRMS (APCI) calcd for C₁₆H₂₀F₂NO₄ [M+H]⁺: 328.14; found, 328.46; T_{R}=2.58 min.

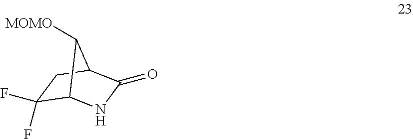

(1S,4S,7R)-6,6-Difluoro-7-(methoxymethoxy)-2-azabicyclo[2.2.1]heptan-3-one (23). To a stirred solution of 22 (900 mg, 2.75 mmol) in CH₃CN (40 mL) was added an aqueous solution of ceric ammonium nitrate (4.52 g in 12 mL water, 8.26 mmol) at room temperature. The resulting mixture was stirred for 2 h until the starting material was completely disappeared. The residue was extracted with EtOAc (25 mL×3). The combined organic layers were separated, washed with sat. Na₂CO₃ solution and brine, dried over Na₂SO₄, and concentrated under vacuum. The crude product was purified via Combi-Flash chromatography (EtOAc-hexane: 0-100%) to obtain 23 (340 mg, 60%) as a light yellow solid. ¹H NMR (500 MHz, CDCl₃) δ 5.91 (s, 1H), 4.70 (s, 2H), 4.29-4.16 (m, 1H), 3.83 (q, J=2.0 Hz, 1H), 3.41 (s, 3H), 2.84 (dt, J=3.9, 1.9 Hz, 1H), 2.62 (dddd, J=18.3, 13.6, 8.9, 4.4 Hz, 1H), 2.13 (dddd, J=15.8, 13.8, 6.2, 1.8 Hz, 1H). ¹³C NMR (126 MHz, CDCl₃) δ 175.0, 128.2 (dd, J=263, 253 Hz), 96.2, 81.7 (d, J=3.3 Hz), 60.5 (dd, J=30.5, 21.6 Hz), 56.24 (d, J=1.8 Hz), 48.66 (dt, J=4.1, 1.5 Hz), 34 11 (dd, J=25.7, 24.2 Hz). LRMS (APCI) calcd for C₈H₁₂F₂NO₃ [M+H]⁺: 208.08; found, 208.07; T_{R}=0.79 min.

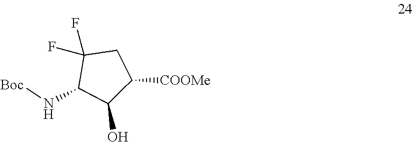 [SMILES: COC(=O)[C@H]1CC(F)(F)[C@@H](NC(=O)OC(C)(C)C)[C@@H]1O]

(1S,2R,3S)-Methyl 3-((tert-Butoxycarbonyl)amino)-4,4-difluoro-2-hydroxycyclo-pentanecarboxylate (24). (i) 23 was dissolved in HCl methanol solution (1.2 M) at room temperature. Then the resulting mixture was heated at 85° C. overnight in a pressure tube. After the completion of the reaction, the excess solvent was removed under vacuum, and then the residue was neutralized with sat. NaHCO₃ solution and extracted with EtOAc (25 mL×3). The combined organic layers were separated, washed with brine, dried over Na₂SO₄, and concentrated under vacuum. The crude product was obtained as a light yellow solid (160 mg, 70%) and used directly into next step without further purification. (ii) To a stirred solution of the intermediate (160 mg, 0.82 mmol) in MeOH (10 mL) was added Boc₂O (268 mg, 1.23 mmol) at room temperature. Then the resulting mixture was stirred at room temperature overnight. After the completion of the reaction, the excess solvent was removed under vacuum, and then the residue was quenched with water and extracted with EtOAc (25 mL×3). The combined organic layers were separated, washed with brine, dried over Na₂SO₄, and concentrated under vacuum to obtain 24 as an off-white solid (230 mg, 95%) and used directly to the next step without further purification. ¹H NMR (500 MHz, CDCl₃) δ 5.07 (s, 1H), 4.19 (t, J=8.4 Hz, 1H), 4.13 (s, 1H), 4.09-3.98 (m, 1H), 3.76 (s, 3H), 2 92 (q, J=9.4 Hz, 1H), 2.67-2.39 (m, 2H), 1.46 (s, 9H). ¹³C NMR (126 MHz, CDCl₃) δ 172.5, 156.7, 124.4 (t, J=254 Hz), 81.2, 77.8 (d, J=6.9 Hz), 62.9 (dd, J=25.2, 17.9 Hz), 52.6, 44.6, 35.0 (t, J=26.0 Hz), 28.2 (3C). LRMS (APCI) calcd for C₇H₁₂F₂NO₃ [M-Boc+2H]⁺: 196.08; found, 196.03; T_{R}=2.20 min.

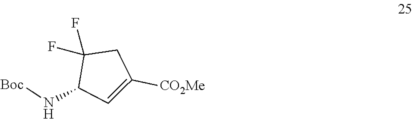 [SMILES: COC(=O)C1=C[C@H](NC(=O)OC(C)(C)C)C(F)(F)C1]

(S)-Methyl 3-((tert-Butoxycarbonyl)amino)-4,4-difluorocyclopent-1-enecarboxy-late (25). To a stirred solution of 24 (230 mg. 0.78 mmol) in THF (5 mL) was added Burgess reagent (580 mg, 2.44 mmol) at room temperature under an argon atmosphere. Then the resulting mixture was heated to reflux for 4 h. After the completion of the reaction, the solution was quenched with water and extracted with EtOAc (25 mL×3). The combined organic layers were separated, washed with brine, dried over Na₂SO₄, and concentrated under vacuum. The crude product was purified via Combi-Flash chromatography (EtOAc/hexane: 0-100%) to obtain 25 (110 mg, 49%) as an off-white solid. LRMS (APCI) calcd for C₇H₁₀F₂NO₂ [M-Boc+2H]⁺: 178.07; found, 177.86; T_{R}=2.65 min.

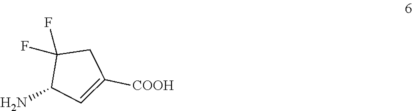 [SMILES: N[C@H]1C=C(C(=O)O)CC1(F)F]

(S)-3-Amino-4,4-difluorocyclopent-1-enecarboxylic acid hydrochloric acid salt (SS-1-148, 6). To a stirred solution of 25 (200 mg, 0.72 mmol) in acetic acid (10 mL) was added 4N HCl (10 mL) at room temperature. The resulting mixture was heated at 70° C. overnight. After the completion of the reaction, the excess solvent was removed under vacuum. The crude product was purified via Combi-Flash chromatography (C18 Reverse Column, CH₃CN/H₂O: 0-5%) to obtain 6 as a white powder HCl salt (72 mg, 50%). ¹H NMR (500 MHz, CD₃OD) δ 6.58 (s, 1H), 4.77-4.67 (m, 1H), 3.29-3.16 (m, 2H) ¹³C NMR (126 MHz, CD₃OD) δ 165.2, 140.0, 133.4, 128.0 (t, J=254.8 Hz), 61.0 (dd, J=35.5, 20.7 Hz), 41.64 (t, J=27.1 Hz). HRMS (ESI) calcd for C₆H₈FNO₂ [M+H]⁺: 164.0518; found, 164.0517.

Expression and Purification of hOAT.

hOAT was expressed and purified using previously published protocols.³ Briefly, E. coli BL21(DE3) cells containing the pMAL-t-hOAT plasmid were incubated at 37° C. with shaking in Lysogeny Broth (LB) medium supplemented with 100 μg/mL ampicillin. When the culture OD₆₀₀ reached a value of 0.7, expression of the MBP-t-hOAT fusion protein was induced by the addition of 0.3 mM isopropyl β-D-1-thiogalactopyranoside and incubated for an additional 16-18 h at 25° C. Cells were harvested by centrifugation, washed with buffer A comprised of 20 mM Tris-HCl, 200 mM NaCl, and 100 μM PLP, pH 7.4, and flash-frozen in liquid nitrogen and stored at −80° C. The frozen cell pellet was then thawed, sonicated in buffer A, and centrifuged at 40,000×g for 20 min. The resulting supernatant was loaded onto an amylose affinity column pre-equilibrated with buffer A. The column was washed thoroughly, and the MBP-t-hOAT fusion protein was eluted from the column with 10 mM maltose. Fractions containing the fusion protein were combined and treated with TEV protease to remove the MBP tag. The cleaved hOAT protein was collected and concentrated using a centrifugal filter. The protein was then further purified by size exclusion chromatography using a HiLoad Superdex-200PG column. The column was pre-equilibrated, and the protein eluted in buffer containing 50 mM HEPES, 100 μM PLP, and 300 mM NaCl, pH 7.5.

### Aminotransferases and Coenzymes for Kinetic Studies.

All reagents for enzyme purification and assays were purchased from Sigma-Aldrich (St. Louis, MO, USA). Human OAT (0.672 mg/mL) was purified from E. coli BL21 (DE3) cells following the procedure described above, and coenzyme human recombinant pyrroline 5-carboxylate reductase (PYCRI) were expressed, grown, and purified according to a literature procedure.⁴ γ-Aminobutyric acid aminotransferase (GABA-AT, 0.4 mg/mL) was purified from pig brain following a procedure described previously⁵, and coenzyme succinic semialdehyde dehydrogenase (SSDH) was purified from GABase (catalog No. G7509-25UN, Sigma-Aldrich), a commercially available mixture of SSDH and GABA-AT, using a known procedure.⁶ Commercially available L-aspartate aminotransferase (Asp-AT) from the porcine heart (catalog No. G2751-2KU, Sigma-Aldrich) and the desired assay coenzyme malic dehydrogenase from porcine (catalog No. M2634-5KU, Sigma-Aldrich), and L-alanine aminotransferase (Ala-AT) from the porcine heart (catalog No. G8255-200UN, Sigma-Aldrich) and the desired assay coenzyme lactate dehydrogenase from porcine (catalog No. 427211-50KU, Sigma-Aldrich) were used directly without further purification. All the enzyme assays for the inactivation, partition ratio, and dialysis experiment were recorded on a Synergy H1 hybrid multimode microplate reader (Biotek, USA) with transparent 96-well plates.

Evaluation of Compounds as Time-Dependent Inhibitors of hOAT.

Time-dependent inactivation against hOAT was performed using an optimized procedure published previously.⁷ 10 μL of hOAT (0.025 mg/mL) was preincubated with 10 μL of various concentrations of tested compounds in 100 mM potassium pyrophosphate buffer (pH 8.0, 5 mM α-ketoglutarate, and 5 mM β-mercaptoethanol) at 25° C. in a 96-well plate. At time intervals, 80 μL of assay solution containing PYCR¹ (0.5 μg), 1 mM NADH, and 25 mM L-ornithine in the abovementioned potassium pyrophosphate buffer were added to the incubation mixture and assayed for OAT activity at 37° C. for 30 min. The change in UV absorbance at 340 nm at 37° C. based on the conversion of NADH to NAD was monitored by a microplate reader. All assays were performed in duplicate, and the remaining OAT activity at each preincubation time at each inhibitor concentration was averaged. The natural logarithm of the percentage of the remaining OAT activity was plotted against the preincubation time at each inhibitor concentration to obtain the k_{obs} (slope) value for each concentration. The k_{obs} value is the rate constant describing the inactivation at each inhibitor concentration. k_{obs} is replotted against the inhibitor concentration using nonlinear regression analysis using GraphPad Prism 8.0. K₁ and k_{inact} values were estimated from equation (1)

$${k}_{obs} = \frac{{k}_{inact} \times [ I ]}{{k}_{1} + [ I ]} \qquad ( 1 )$$

where k_{inact} is the maximal rate of inactivation, K₁ is the inhibitor concentration required for half-maximal inactivation, and [I] is the preincubation concentration of the tested compound.

### Evaluation of Compounds as Reversible Inhibitors of GABA-AT.

Inhibition constants (Kᵢ) were determined by monitoring the GABA-AT activity in the presence of 0-20 mM concentrations of the tested compounds using a coupled assay with SSDH. 96-well plates were loaded with 80 μL of an assay mixture containing excess SSDH, 1 mM NADP⁺, 11.1 mM GABA, and 10 μL of various concentrations of the tested compounds in 50 mM potassium pyrophosphate buffer (pH 8.5, 5 mM α-ketoglutarate, and 5 mM β-mercaptoethanol). After incubation of the mixture at 25° C. for 1 min, 10 μL of GABA-AT (0.03 mg/mL) in the abovementioned potassium pyrophosphate buffer was added to the 96-well plate. The microplate was shaken at 25° C. for 1 min, and the remaining enzyme activity was determined by observing the change in absorbance at 340 nm at 25° C. based on the conversion of NADP⁺ to NADPH. Half-maximal inhibitory concentration (IC₅₀) values were obtained using nonlinear regression analysis using GraphPad Prism 8.0. Subsequent Kᵢ values were determined using equation (2) All assays were performed in duplicate.

$${K}_{i} = \frac{{IC}_{50}}{1 + \frac{[ S ]}{Km}} \qquad ( 2 )$$

where [S] is the final concentration of substrate GABA (8.88 mM), Kₘ is the Michaelis-Menten constant, which was determined to be 2.60 mM for this batch of GABA-AT isolated from pig brain.

### Inhibition of Asp-AT and Ala-AT by SS-1-148.

96-well plate was loaded with 90 μL of an assay mixture containing 100 mM

potassium phosphate at pH 7.4, 5.55 mM α-ketoglutarate, 1.11 mM NADH, 5.55 mM Z-aspartate, 5.55 units of malic dehydrogenase, and various concentrations of SS-1-148. After incubation of the mixture at room temperature for a few minutes, 10 μL of Asp-AT (2.0 units/mL in 100 mM potassium phosphate at pH 7 4) was added. The plate was shaken at room temperature for 1 min, and the absorbance was measured at 340 nm every 10 s for 90 min based on the conversion of NADH to NAD⁺. The remaining Asp-AT activity percentage was calculated according to the corresponding concentrations. All assays were performed in duplicate. The assay conditions for determining the inhibition of Ala-AT by SS-1-148 were identical to that with L-aspartate, except L-alanine was used as the substrate, and lactate dehydrogenase was used as the coenzyme.

### Dialysis Assay

The dialysis experiment was conducted using previous protocols.⁸⁻¹⁰

### Partition Ratio Experiment

The partition ratio was calculated using previous protocols.⁸⁻¹⁰

### Fluoride Ion Release Assay

The fluoride ion release assay was conducted using previous protocols.¹⁰ The final concentration of enzyme in the sample was determined via BCA protein assay kit (Pierce, cat: 23225). A calibration curve of voltage (V, mV) was generated from varying concentrations of NaF (F, μM). For accurate detection of fluoride ion concentration, 10 μM of fluoride ion was added to each control and sample The number of fluoride ions released per active site was calculated by the ratio of the fluoride ion release concentration and enzyme concentration.

### Native Protein Mass Spectrometry

Treated and unmodified hOAT samples were separately desalted by dialysis against 100 mM NH₄(Ac) in D-Tube™ Dialyzer Mini (MWCO₁₂-14 KDa; Lot #: 3455246; Millipore) for 96 h at 4° C. Desalted samples were separately introduced into a Q-Exactive Ultra High Mass Range (UHMR) (Thermo Fisher Scientific) mass spectrometer with a Nanospray Flex Ion Source (Thermo Fisher Scientific) with spray voltages between +1500 to +2000 V. The ion transfer tube was set to 310° C. The mass spectrometer was run in +ESI mode, with full scan data collected from 4000-10,000 m/z with 200 microscans and an Orbitrap resolution of 35,000 or 17,500 (at 200 m/z) and a target AGC of 1e6 charges. The S-lens RF level was set to 200% and extended trapping set to 100. To eject adducts from protein dimers, HCD collisional activation was applied with a normalized collisional energy (NCE) set to 5-15. Raw spectra from each experiment were summed across multiple scans. Mass deconvolution was performed using mMass¹¹ and/or UniDec¹² to generate zero-charge masses and associated mass standard deviations.

### Intact Protein Mass Spectrometry and Metabolomics

Treated and unmodified purified hOAT samples were desalted ten times with Optima-grade water (Fisher) on Amicon Ultra 10 KDa molecular weight spin filters (Millipore). To chromatographically resolve protein. 0.5 μg of protein was loaded onto a 3 cm PLRP-S (Agilent) trap column using a Dionex Ultimate3000 liquid chromatography system (Thermo Fisher). The protein analyte was washed with a 10-min isocratic gradient of 10% Solvent B (95% acetonitrile/5% H₂O/0.2% formic acid) and 90% Solvent A (5% acetonitrile/95% H_{20/0.2}% formic acid). Protein was resolved on an in-house made 75 μm ID×15 cm long nanopore capillary column packed with PLRP-S resin (Agilent). The LC system was operated at a flow rate of 0.3 μL/min at the following gradient: 0-10 min 10% Solvent B; 10-12 min to 40% Solvent B, 12-22 min to 90% Solvent B; 22-24 min at 90% Solvent B; 24-26 min to 10% Solvent B; 26-30 min isocratic at 10% Solvent B. hOAT samples were introduced into a Thermo Fischer Orbitrap Fusion Lumos or Eclipse mass spectrometer, and full MS data were acquired as previously described.⁸ Small molecule and metabolite masses were identified and characterized by positive and negative mode high-resolution LC-MS/MS on a Q-Exactive Orbitrap mass spectrometer (Thermo) as previously described.^{2, 8, 13}

Crystallization and Crystal Soaking of hOAT with SS-1-148

hOAT crystal soaking. The holoenzyme crystals were first grown via a hanging drop vapor diffusion method. Each drop contained 2 μL of protein and 2 μL of well solution. The best crystallization condition contained 10% PEG 8000, 200 mM NaCl, 10% glycerol. Once the holoenzyme crystals reached their maximum size, 2 μL of 16 mM SS-1-148 was added to the drop with crystals. Within the first five minutes of SS-1-148 addition, the hOAT crystals turned their color from yellow to blue. The crystals were soaked for 1 h, transferred into cryoprotective solution (well solution supplemented with 30% glycerol), and then flash-frozen in liquid nitrogen.

hOAT co-crystallization. After purification, hOAT was buffer exchanged into crystallization buffer (50 mM Tricine pH 7.8) supplied with 5 mM α-ketoglutarate. Then the protein was concentrated to 6 mg/mL. Previously reported crystallization condition⁸ was optimized using the hanging drop vapor diffusion method by varying PEG 6000 (8-12%), NaCl (100-250 mM), glycerol (0%-10%) with 100 mM Tricine pH 7.8 was kept constant as the buffer. For each hanging drop, 2 μL of protein solution was mixed with an equal volume of well solution and 0.5 μL of 10 mM SS-1-148. The crystals with the best morphology and size grew in a final condition containing 10% PEG 8000, 200 mM NaCl, 10% glycerol. Crystals were transferred to a cryo-protectant solution (well solution supplemented with 30% glycerol) and later flash-frozen in liquid nitrogen.

X-ray diffraction and data processing. Monochromatic X-ray diffraction data were collected at the LS-CAT beamline 21-ID-D at the Advanced Photon Source at Argonne National Laboratory. Data were collected at a wavelength of 1.127 Å and a temperature of 100 K using a Dectris Eiger 9M detector. Data sets were processed and analyzed with autoPROC software.¹⁴

Model building and refinement. The hOAT structure was solved by molecular replacement using PHASER¹⁵ in Phenix. The starting search model was the previously published structure of hOAT (PDB code: 1OAT¹⁶). The model building and refinement were accomplished in Coot¹⁷ and Phenix¹⁸, respectively, as an iterative process until the lowest possible R_{free}/R factor values were attained. Structural depiction figures were prepared using UCSF Chimera.¹⁹

### Transient State Methods.

The reaction of SS-1-148 with hOAT was observed in a transient state using a Hitech Scientific (TgK) stopped-flow spectrophotometer in combination with charged coupled device detection (260-800 nm). hOAT (12.68 μM) was reacted with varied concentrations (125, 251, 502, 1004, 2008, 4016, 8032 μM) of SS-1-148 in the buffer (pH 7.5) containing 50 mM HEPES and 200 mM NaCl at 10° C. For each concentration of the inhibitor, CCD spectral datasets were collected in duplicate for 50 sec, and the duplicates were averaged For the highest concentration of inhibitor (8 mM) duplicate CCD datasets were acquired for two timeframes (0.0025-12.4 sec and 0.0025-2480 sec). Duplicate datasets for anyone timeframe were averaged and then spliced together at 12 sec to form one dataset with sufficient time resolution to adequately describe rapid and slow processes. The hybrid dataset was deconvoluted based on a linear four-species model using the Spectrafit singular value decomposition module of KinTek Explorer software. In the process, the rate constants were fixed to the values measured using analytical fits to data extracted for single wavelengths that assume successive first-order processes according to the equations (3), (4), and (5). Abs is absorbance, Aₓ are the amplitudes associated with each observed phase, kₓ are the corresponding rate constants, and C is the absorbance endpoint.

Abs=A₁(e^{-k}^{_{1obs}}^{t})+A₂(e^{-k}^{₂}^{t})+A₃(e^{-k}^{₃}^{t})+C (3)

Abs=A₁(e^{-k}^{₁}^{t})+A₂(e^{-k}^{₂}^{t})+C (4)

K_{1obs}=k₁[I] (5)

### Gibb's Free Energy Calculation

MOPAC 2016 is a computational chemistry software that is based on the concept of quantum theory and thermodynamics, using some concepts of advanced mathematics. It is a semiempirical molecular orbital package used for the study of solid state, nanostructures, molecular structures and their reactions.²⁰⁻²¹ In this context, MOPAC 2016 has been used for setting the molecular geometries of each tautomeric forms of M7 (FIG. 14) followed by optimization through the PM7 semiempirical method. Solvation by water molecules was not considered for the calculation. A combination of molecular mechanics energy, polar and nonpolar energies. and entropy have been considered for the resulting binding free energy of each tautomer presented on FIG. 14. Enthalpy (ΔH^{o}) and entropy (ΔS^{o}) contribute to the ΔG^{o} value of each tautomer, also determined by the Gibb's free energy equation: ΔG^{o}=ΔH^{o}−TΔS^{o}.

### Theoretical pKₐ Calculations

The geometries of the neutral and deprotonated species of M1, M1′, and M1″ were fully optimized using the DFT B3LYP/6-31G** level of theory. For all of the investigated compounds, the gas-phase Gibbs free energy changes (ΔG^{o}_{g}) of compounds were calculated using Gaussian09 software.²² The solvation free energies were calculated by applying polarizable continuum model (PCM), using the same level of theory and basis set (B3LYP/6-31G**) which was used for geometry determination in the gas phase. The PCM calculations were used with the UAHF atomic radii when building the solvent cavity calculates the Gibb's free energy of solvation The pKₐ values were obtained applying the following equations (6), (7), and (8) and the thermodynamic cycle A presented by Ghalami-Choobar and coworkers.²³

ΔG_{aq}^{o}=ΔG_{g}^{o}+δΔGₛ^{o} (6)

ΔG_{aq}^{o}=−2.303RT log Kₐ (7)

ΔG_{aq}^{o}=ΔGₛ^{o}(A⁻)+ΔGₛ^{o}(H⁺)−ΔGₛ^{o}(AH)+G_{g}^{o}(A⁻)+G_{g}^{o}(H⁺)−G_{g}^{o}(AH) (8)

### Electrostatic Potential (ESP) Charge Calculation

The three-dimensional (3D) molecular models of M1, M1′, and M1″ were built up using Spartan'14 software (Wavefuction, Inc., 2014). The build structures were refined by molecular mechanics using Merck molecular force field (MMFF₉₄). Then, with the lowest energy conformer selected, the equilibrium geometry and molecular orbitals were calculated using Hartree-Fock (HF) at the 6-31G* level of theory. Spartan'14 was also used to generate the electron density and electrostatic potential maps.

### ABBREVIATION

Boc₂O, di-tert-butyl dicarbonate, Deoxo-Fluor, bis(2-methoxyethyl)aminosulfur trifluoride: DMPK, drug metabolism and pharmacokinetics; DIPEA, N,N-diisopropylethylamine; DBDMH, 1,3-dibromo-5,5-dimethylhydantoin; DMF, dimethylformamide; DMSO, dimethyl sulfoxide; DCM, dichloromethane; THF, tetrahydrofuran; TEA, triethylamine; TBAI, tetra-n-butylammonium iodide

### REFERENCES

1. Lu, H.; Silverman, R. B., Fluorinated conformationally restricted gamma-aminobutyric acid aminotransferase inhibitors. J Med Chem 2006, 49 (25), 7404-12.
2. Shen, S.; Doubleday, P. F.; Weerawarna, P. M.; Zhu, W.; Kelleher, N. L.; Silverman, R. B., Mechanism-Based Design of 3-Amino-4-Halocyclopentenecarboxylic Acids as Inactivators of GABA Aminotransferase. ACS Med Chem Lett 2020, 11 (10), 1949-1955.
3. Mascarenhas, R.; Le, H. V.; Clevenger, K. D.; Lehrer, H. J.; Ringe, D.; Kelleher, N. L.; Silverman, R. B.; Liu, D., Selective Targeting by a Mechanism-Based Inactivator against Pyridoxal 5′-Phosphate-Dependent Enzymes: Mechanisms of Inactivation and Alternative Turnover. Biochemistry 2017, 56 (37), 4951-4961.
4. Christensen, E. M.; Patel, S. M.; Korasick, D. A.; Campbell, A. C.; Krause, K. L.; Becker, D. F.; Tanner, J. J., Resolving the cofactor-binding site in the proline biosynthetic enzyme human pyrroline-5-carboxylate reductase 1. J Biol Chem 2017, 292 (17), 7233-7243.
5. Koo, Y. K.; Nandi, D.; Silverman, R. B., The multiple active enzyme species of gamma-aminobutyric acid aminotransferase are not isozymes. Arch Biochem Biophys 2000, 374 (2), 248-54.
6. Silverman, R. B.; Bichler, K. A.; Leon, A. J. J. Am. Chem. Soc. 1996, 118 (6). 1241-52.
7. Juncosa, J. I.; Lee, H.; Silverman, R. B., Two continuous coupled assays for ornithine-delta-aminotransferase. Anal Biochem 2013, 440 (2), 145-9.
8. Moschitto, M. J.; Doubleday, P. F.; Catlin, D. S.; Kelleher, N. L.; Liu, D.; Silverman, R. B., Mechanism of Inactivation of Ornithine Aminotransferase by (1S,3S)-3-Amino-4-(hexafluoropropan-2-ylidenyl)cyclopentane-1-carboxylic Acid. J Am Chem Soc 2019, 141 (27), 10711-10721.
9. Juncosa, J. I.; Takaya, K.; Le, H. V.; Moschitto, M. J.; Weerawarna, P. M.; Mascarenhas, R.; Liu, D. L.; Dewey, S. L.; Silverman, R. B., Design and Mechanism of (S)-3-Amino-4-(difluoromethylenyl)cyclopent-1-ene-1-carboxylic Acid, a Highly Potent gamma-Aminobutyric Acid Aminotransferase Inactivator for the Treatment of Addiction. J Am Chem Soc 2018, 140 (6), 2151-2164.
10. Lee, H.; Doud, E. H.; Wu, R.; Sanishvili, R.; Juncosa, J. I.; Liu, D. L.; Kelleher, N. L.; Silverman, R. B., Mechanism of Inactivation of gamma-Aminobutyric Acid Aminotransferase by (1S,3S)-3-Amino-4-difluoromethylene-1-cyclopentanoic Acid (CPP-115). J Am Chem Soc 2015, 137 (7), 2628-2640.
11. Niedermeyer, T. H.; Strohalm, M., mMass as a software tool for the annotation of cyclic peptide tandem mass spectra. PLOS One 2012, 7 (9), e44913.
12. Marty, M. T.; Baldwin, A. J.; Marklund, E. G.; Hochberg, G. K.; Benesch, J. L.; Robinson, C. V., Bayesian deconvolution of mass and ion mobility spectra: from binary interactions to polydisperse ensembles. Anal Chem 2015, 87 (8), 4370-6.
13. Zhu, W.; Doubleday, P. F.; Catlin, D. S.; Weerawama, P. M.; Butrin, A.; Shen, S.; Wawrzak, Z.; Kelleher, N. L.; Liu, D.; Silverman, R. B., A Remarkable Difference That One Fluorine Atom Confers on the Mechanisms of Inactivation of Human Ornithine Aminotransferase by Two Cyclohexene Analogues of gamma-Aminobutyric Acid. J Am Chem Soc 2020, 142 (10), 4892-4903.
14. Vonrhein, C.; Flensburg, C.; Keller, P.; Sharff, A.; Smart, O.; Paciorek, W.; Womack, T.; Bricogne, G., Data processing and analysis with the autoPROC toolbox. Acta Crystallogr D Biol Crystallogr 2011, 67 (Pt 4), 293-302.
15. McCoy, A. J.; Grosse-Kunstleve, R. W.; Adams. P. D.; Winn, M. D.; Storoni, L. C.; Read, R. J., Phaser crystallographic software. J Appl Crystallogr 2007, 40 (Pt 4), 658-674.
16. Shen, B. W.; Hennig, M.; Hohenester, E.; Jansonius, J. N.; Schirmer, T., Crystal structure of human recombinant ornithine aminotransferase. J Mol Biol 1998, 277 (1), 81-102.
17. Emsley, P.; Lohkamp, B.; Scott, W. G.; Cowtan, K., Features and development of Coot. Acta Crystallogr D Biol Crystallogr 2010, 66 (Pt 4), 486-501.
18. Liebschner, D.; Afonine, P. V.; Baker, M. L.; Bunkoczi, G.; Chen, V. B.; Croll, T. I.; Hintze, B.; Hung, L. W.; Jain, S.; McCoy, A. J.; Moriarty, N. W.; Oeffner, R. D.; Poon, B. K.; Prisant, M. G.; Read, R. J.; Richardson, J. S.; Richardson, D. C.; Sammito, M. D.; Sobolev, O. V.; Stockwell, D. H.; Terwilliger, T. C.; Urzhumtsev, A. G.; Videau, L. L.; Williams, C. J.; Adams, P. D., Macromolecular structure determination using X-rays, neutrons and electrons: recent developments in Phenix. Acta Crystallogr D Struct Biol 2019, 75 (Pt 10), 861-877.
19. Pettersen, E. F.; Goddard, T. D.; Huang, C. C.; Couch, G. S.; Greenblatt. D. M.; Meng. E. C.; Ferrin, T. E., UCSF Chimera—a visualization system for exploratory research and analysis. J Comput Chem 2004, 25 (13), 1605-12.
20. Dewar, M. J. S.; Thiel, W., Ground states of molecules. 39. MNDO results for molecules containing hydrogen, carbon, nitrogen, and oxygen. Journal of the American Chemical Society 1977, 99 (15), 4907-4917.
21. Stewart, J. J. P., Optimization of parameters for semiempirical methods VI: more modifications to the NDDO approximations and re-optimization of parameters. Journal of Molecular Modeling 2013, 19 (1), 1-32.
22. Gaussian 09, Revision A. 02, M. J. Frisch, G. W. Trucks, H. B. Schlegel, G. E. Scuseria, M. A. Robb, J. R. Cheeseman, G. Scalmani, V. Barone, G. A. Petersson, H. Nakatsuji, X. Li, M. Caricato, A. Marenich, J. Bloino, B. G. Janesko, R. Gomperts, B. Mennucci, H. P. Hratchian, J. V. Ortiz, A. F. Izmaylov, J. L. Sonnenberg, D. Williams-Young, F. Ding, F. Lipparini, F. Egidi, J. Goings, B. Peng, A. Petrone, T. Henderson, D. Ranasinghe, V. G. Zakrzewski, J. Gao, N. Rega, G. Zheng, W. Liang, M. Hada, M. Ehara, K. Toyota, R. Fukuda, J. Hasegawa, M. Ishida, T. Nakajima, Y. Honda, O. Kitao, H. Nakai, T. Vreven, K. Throssell, J. A. Montgomery, Jr., J. E. Peralta, F. Ogliaro, M. Bearpark, J. J. Heyd, E. Brothers, K. N. Kudin, V. N. Staroverov, T. Keith, R. Kobayashi, J. Normand, K. Raghavachari, A. Rendell, J. C. Burant, S. S. Iyengar, J. Tomasi, M. Cossi, J. M. Millam, M. Klene, C. Adamo, R. Cammi, J. W. Ochterski, R. L. Martin, K. Morokuma, O. Farkas, J. B. Foresman, and D. J. Fox, Gaussian, Inc., Wallingford CT, 2016.
23. Ghalami-Choobar, B.; Dezhampanah, H.; Nikparsa, P.; Ghiami-Shomami, A., Theoretical calculation of the pKa values of some drugs in aqueous solution. International Journal of Quantum Chemistry 2012, 112 (10), 2275-2280.

In accordance with this disclosure, various other compounds, varied structurally, stereochemically and/or configurationally, are available through such incorporated synthetic procedures and techniques or straight-forward modifications thereof, such modifications as would also be known and understood by those skilled in the art and made aware of this invention, such procedures, techniques and modifications limited only by the commercial or synthetic availability of any corresponding reagent or starting material.

1. A compound of the following formula or a dissociated form, a non-protonated form, a zwitterion form, or a salt thereof: 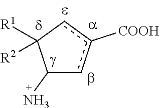
wherein a double bond is present between the a and & carbons or between the α and β carbons, and
wherein each of R¹ and R² is independently selected from a halogen such as F, Cl, Br, and I.

2. The compound of claim 1 in zwitterion form comprising an ammonium moiety and a carboxylate moiety.

3. The compound of claim 1, wherein the double bond is between the α and ε carbons.

4. The compound of claim 1, wherein the double bond is between the α and β carbons.

5. The compound of claim 1, wherein at least one of R¹ and R² is F.

6. The compound of claim 5, wherein the compound is a salt comprising a substituent selected from an ammonium substituent, a carboxylate substituent, and a combination thereof.

7. The compound of claim 6, wherein the ammonium salt has a counter ion that is the conjugate base of a protic acid.

8. The compound of claim 1 in a pharmaceutical composition comprising a pharmaceutically-acceptable carrier component.

9. The compound of claim 1 of a formula: 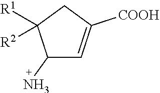
wherein at least one of R¹ and R² is F.

10. The compound of claim 9, wherein the compound is a salt comprising a substituent selected from an ammonium substituent, a carboxylate substituent, and a combination thereof.

11. The compound of claim 1 of a formula:

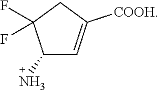 [SMILES: N[C@H]1C=C(C(=O)O)CC1(F)F]

12. The compound of claim 11, wherein the compound is a salt comprising a substituent selected from an ammonium substituent, a carboxylate substituent, and a combination thereof.

13. The compound of claim 1 of a formula:

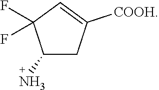 [SMILES: N[C@H]1CC(C(=O)O)=CC1(F)F]

14. The compound of claim 13, wherein the compound is a salt comprising a substituent selected from an ammonium substituent, a carboxylate substituent, and a combination thereof.

15. A pharmaceutical composition comprising: (i) the compound of claim 1; and (ii) a pharmaceutically suitable carrier, diluent, or excipient.

16. A method of modulating human ornithine δ-aminotransferase (hOAT) activity, the method comprising contacting the compound of claim 1 with a medium comprising hOAT, wherein the compound is present in an amount sufficient to modulate hOAT activity.

17. A method of reducing activity of an hOAT expressed by a human cancer, the method comprising contacting the cancer expressing the hOAT with an effective amount of the compound of claim 1 to reduce hOAT activity.

18. A method for treating cancer in a subject in need thereof, the method comprising administering to the subject a therapeutically effective amount of the compound of claim 1.

19. The method of claim 18, wherein the cancer is hepatocellular carcinoma (HCC).

20. The method of claim 18, wherein the cancer is non-small cell lung cancer (NSCLC).

21. The method of claim 18, wherein the cancer is characterized by expression or overexpression of human ornithine δ-aminotransferase (hOAT).

22.-34. (canceled)
